SEQUENCE LISTING (29 sequences)

SEQ ID NO: 1 - DNA, 2613 bp, Clostridium autoethanogenum
atgaaagttacaaacgtagaagaactaatgaaaagactagaagaaataaaggatgctcaaaagaaatttgctacatatactcaagaacaagtggatgaaatttttagacaagcagctatggcagctaatagtgctagaatagaactagctaaaatggcagtagaagaaagcggaatgggaattgtagaagacaaggttattaaaaatcactttgcttcagaatatatatataacaaatataaggatgaaaaaacctgtggagttttagagagagatgcaggctttggtatagttagaattgcggaacctgtaggagttattgcagcagtagttccaacaactaatccaacatctacagcaatatttaaatcactaatagctttaaaaactagaaatggtataattttttcaccccatccaagggcaaagaaatcaactattgcagcagctaaaatagtacttgacgctgcagttaaagctggtgctcctgaaggaattataggatggatagatgaaccttccattgaactttcacaggtggtaatgggagaagcaaatttaattcttgcaactggtggtccgggtatggttaaggctgcctattcttcaggcaaacctgctgtgggagttggtccaggtaacacacctgctgtaattgatgaaagtgccgacattaaaatggcagtaaattcaatattactatcaaaaacttttgataatggtatgatttgtgcctcagagcagtcagtaatagttttagactcaatatatgaggaagttaaaaaagaatttgcttataggggtgcttatatattaagtaaggatgaaacagataaggttggaaaaataattttaaaaaatggagccttaaatgcaggtattgtaggacaacctgcttttaaaatagcacagctggcaggagtggatgtaccagaaaaagctaaagtacttataggagaggtagaatcggtagaacttgaagaaccattttctcatgaaaagttatctccagttttagctatgtacagggcaagaaattttgaggatgccattgcaaaaactgataaactggttagggcaggtggatttggacatacatcttcattgtatataaatccaatgacagaaaaagcaaaagtagaaaaatttagtactatgatgaaaacatcaagaactataattaacacaccttcatcccaaggtggtataggtgatatatataactttaaactagctccttctttgacattaggctgcggttcctgggggggaaattctgtatccgaaaatgttgggcctaaacatttattaaacataaaaagtgttgctgagaggagagaaaatatgctttggtttagagtacctgaaaaggtttatttcaaatatggtagtcttggagttgcattaaaagagttaaaagttatgaataagaagaaagtatttatagtaacagataaagttctttatcaattaggttatgtggacaaagttacaaaagttcttgaggaactaaaaatttcctataaggtatttacagatgtagaaccagatccaacccttgctacagctaaaaaaggtgcagcagaactgctttcctatgaaccggatacaattatatcagttggtggtggttcagcaatggatgcagctaagatcatgtgggtaatgtatgagcatccagaagtaaaatttgaagatttagctatgagatttatggatataagaaagagagtatatgttttccctaagatgggagaaaaggcaatgatgatttcagtagcaacatccgcaggaacagggtcggaagttactccatttgcagtaatcactgatgaaaaaacaggagctaaatatccattagctgattatgaactaactccagacatggctatagttgatgcagaacttatgatgggaatgccaagaggacttacagcagcttcgggtatagatgcattaacccatgcactggaggcgtatgtgtcaataatggctacagaatttaccaatggattagcccttgaagcagtaaagttgatatttgaatatttaccaaaagcttatacagaaggtacaactaatgtaaaggcaagagaaaagatggctcatgcttcatgtattgcaggtatggcctttgcaaatgcatttttaggggtatgccactctatggcacataaattgggagcacagcatcacataccacatggaattgccaatgcacttatgatagatgaagttataaaattcaatgctgtagatgatccaataaaacaagctgcatttccccaatacgagtatccaaatgctaggtatagatatgctcagatagctgattgtctgaacttgggaggaaatacagaagaggaaaaggtacaactattaataaatgctatagatgatttaaaagctaagttaaatattccagaaactataaaagaagcaggagtttcagaagataaattctatgctactttagataaaatgtcagaattagcttttgatgatcagtgtacaggagctaatccaagatatccactgataagtgaaataaaacaaatgtatataaatgtttttgataaaaccgaaccaattgtagaagatgaagaaaagtaa

SEQ ID NO: 2 - DNA, 2613 bp, Artificial sequence
  codon-optimized gene of Clostridium autoethanogenum
atgaaagtaactaatgtagaagaattaatgaaaagattagaagaaataaaagatgcacaaaaaaaatttgcaacttatactcaagaacaagtagatgaaatatttagacaagcagcaatggcagcaaattctgcaagaatagaacttgcaaaaatggctgtagaagaaagtggaatgggaatagtagaagataaagtaataaaaaatcattttgctagtgaatatatatataataaatataaagatgaaaaaacttgtggtgtacttgaaagagatgcaggatttggaatagtaagaatagcagaacctgttggagtaatagcagctgtagtacctactactaatcctactagtactgcaatatttaaatctttaatagcattaaaaactagaaatggaataatttttagtcctcatcctagagcaaaaaaaagtactatagcagctgcaaaaattgtattagatgcagctgtaaaagctggtgcacctgaaggtataataggatggatagatgaacctagtatagaattaagtcaagtagtaatgggagaagcaaatcttatacttgcaactggtggacctggaatggtaaaagcagcatatagtagtggaaaacctgcagttggagtaggacctggaaatactcctgcagtaatagatgaaagtgcagatataaaaatggcagtaaatagtatattacttagtaaaacttttgataatggaatgatatgtgcaagtgaacaaagtgtaatagtattagatagtatatatgaagaagtaaaaaaagaatttgcatatagaggtgcatatatactttcaaaagatgaaactgataaagtaggaaaaataatacttaaaaatggtgctcttaatgctggaatagtaggacagcctgcatttaaaatagcacaattagctggtgtagatgtacctgaaaaagcaaaagtattaattggagaagtagaaagtgtagaattagaagaaccttttagtcatgaaaaacttagtcctgtattagcaatgtatagagcaagaaattttgaagatgcaatagcaaaaactgataaattagtaagagctggaggatttggacatactagttcattatatataaatcctatgactgaaaaagctaaagttgaaaaatttagtactatgatgaaaactagtagaactataataaatactcctagtagtcaaggtggaattggagatatatataattttaaattagcacctagtttaactttaggatgtggaagttggggaggaaatagtgtatctgaaaatgtaggacctaaacatttattaaatataaaaagtgtagcagaaagaagagaaaatatgctttggtttagagtaccagaaaaagtatattttaaatatggaagtcttggagtagcattaaaagaattaaaagtaatgaataagaaaaaagtatttatagttacagataaagtattatatcaattaggatatgtagataaagttactaaagtacttgaagaattaaaaataagttataaagtatttactgatgtagaacctgatcctactttagcaacagcaaaaaaaggtgctgcagaattattaagttatgaacctgatactataataagtgttggaggtggaagtgcaatggatgcagcaaaaataatgtgggtaatgtatgaacatcctgaagtaaaatttgaagatttagcaatgagatttatggatataagaaaaagagtatacgtatttcctaaaatgggagaaaaagcaatgatgataagtgtagcaactagtgctggaactggaagtgaagtaactccttttgcagtaataactgatgaaaaaacaggtgcaaaatatcctttagcagattatgaattaactcctgatatggcaatagtagatgcagaacttatgatgggaatgcctagaggattaactgcagcaagtggaatagatgcattaactcatgcattagaagcttatgtaagtataatggcaactgaatttactaatggattagcattagaagcagtaaaacttatatttgaatatttacctaaagcatatactgaaggtactactaatgtaaaagcaagagaaaaaatggcacatgcaagttgtatagctggaatggcttttgcaaatgcatttcttggagtatgtcatagtatggcacataaacttggagcacagcatcatatacctcatggaatagcaaatgcattaatgatagatgaagttataaaatttaatgcagtagatgatccaataaaacaagcagcatttcctcaatatgaatatcctaatgcaagatatagatatgcacaaatagcagattgtttaaatcttggaggaaatactgaagaagaaaaagtacaattacttataaatgcaatagatgatttaaaagcaaaacttaatatacctgaaacaataaaagaagctggagtatctgaagataaattttatgcaacattagataaaatgtctgaattagcatttgatgatcaatgtactggtgcaaatcctagatatcctttaataagtgaaataaaacaaatgtatataaatgtatttgataaaactgaacctatagttgaagatgaagaaaaataa

SEQ ID NO: 3 - DNA, 37 bp, Artificial sequence
  Synthetic primer
ccgaattcgtcgacaacagagtttgatcctggctcag

SEQ ID NO: 4 - DNA, 37 bp, Artificial sequence
  Synthetic primer
cccgggatccaagcttacggctaccttgttacgactt

SEQ ID NO: 5 - DNA, 25 bp, Artificial sequence
  Synthetic primer
gaagagaaaatatgctttggtttag

SEQ ID NO: 6 - DNA, 25 bp, Artificial sequence
  Synthetic primer
ctatcattaatgcatttgctattcc

SEQ ID NO: 7 - DNA, 31 bp, Artificial sequence
  Synthetic primer
ggaagtcatatgaaagtcacaacagtaaagg

SEQ ID NO: 8 - DNA, 36 bp, Artificial sequence
  Synthetic primer
tggaagctagcttaaggttgttttttaaaacaattt

SEQ ID NO: 9 - DNA, 32 bp, Artificial sequence
  Synthetic primer
ggaagtcatatgaataaagacacactaatacc

SEQ ID NO: 10 - DNA, 34 bp, Artificial sequence
  Synthetic primer
tggaagctagcttagccggcaagtacacatcttc

SEQ ID NO: 11 - protein, 1170 aa, Clostridium autoethanogenum
MRKMKTMDGNTAAAYISYAFTDVAAIYPITPSSPMAEHVDEWVAKGKKNIFGQQVKVMEMQSEAGASGAVHGSLQAGALTSTYTASQGLLLMIPNMYKIAGELLPGVFHVSARAVAANSLNIFGDHQDVMATRQTGFALFAESSVQQVMDLSAVAHLSAIKGRVPFVNFFDGFRTSHEIQKIELLEYEELAKLVDQKALNDFRRRALNPDHPVTRGTAQNPDIYFQEREVSNTYYEALPEIVEGYMQEMTKLTGREYHLFNYYGAKDAERIIIAMGSVCETVEEVIDYLTTKGEKVGLLTVHLYRPFSIKHFMKYIPKTVKKIAVLDRTKEPGSIGEPLYLDVKNAFYGQEVQPVIVGGRYGLGSKDVLPSHILPVFENLKSDKPKDRFTLSIVDDVTNTSLPVGEDINTTPEGTTACKFWGLGSDGTVGANKSAIKIIGDHTDMYAQGYFAYDSKKSGGITVSHLRFGKSPIKSPYLIDKADFVAVHNQSYVHKYDVLAGLKKGGNFLLNTVWTQEELEKELPASMKKYIANNDIKFYTLNAVKIAQEIGLGGRINMICQSAFFKIANIIPIDDAVKYLKEAVVTSYGKKGQKVVDMNNAAIDKGVNAVVKIDVPASWKDAEDEAAATKELPKFIEKIVNPMNRQEGDKLPVSAFVGMEDGTFPAGTAAYEKRGIAINVPEWQVDKCIQCNQCSFVCPHAAIRPVLTTEEELAKAPQGFEAKDANGAKGLKFTMAISPLDCSGCGNCEDVCPAKEKALVMKPVDTQLSKTEAWDYAVNAVAHKDNPMKDKYSVKASQFEQPLLEFSGACAGCGETPYVKLVTQLFGDRMMIANATGCSSIWGASAPATPYTTNYRGHGPSWANSLFEDNAEYGLGMFLGVKQTRERLQDKIEEALKGSLSAELKAAFEDWIKNFAEGEGTRERADKITALLEKEKGSNDLLNDIYENRDFLVKRSHWIIGGDGWGYDIGYGGVDHVLASNEDVNILVLDTEVYSNTGGQSSKSTPTAAVAKFAASGKKTKKKDLGMMAMSYGYVYVAQISMGADKNQALKAIHEAEAYHGPSLIIAYAPCINHGLRVGMGKSQREAKRAVDCGYWALYRYNPELKEEGKKSFSLDSKEPTTDFKEFLMGEVRYSSLAKQFPDQADALFEKTKKDALQRIAGYKKLDNEQ

SEQ ID NO: 12 - DNA, 3519 bp, Artificial sequence
  codon-optimized gene of Clostridium autoethanogenum
atgagaaaaatgaaaactatggatggaaatactgcagcagcatatataagttatgcatttactgatgtagcagcaatatatcctataactcctagtagtcctatggcagaacatgtagatgaatgggtagcaaaaggaaaaaaaaatatatttggacaacaagtaaaagtaatggaaatgcaaagtgaagctggagcaagtggagcagtacatggaagtttacaagctggagcattaactagtacttatactgcaagtcaaggattattattaatgatacctaatatgtataaaatagctggagaattattacctggagtatttcatgtaagtgcaagagcagtagcagcaaatagtttaaatatatttggagatcatcaagatgtaatggcaactagacaaactggatttgcattatttgcagaaagtagtgtacaacaagtaatggatttaagtgcagtagcacatttaagtgcaataaaaggaagagtaccttttgtaaatttttttgatggatttagaactagtcatgaaatacaaaaaatagaattattagaatatgaagaattagcaaaattagtagatcaaaaagcattaaatgattttagaagaagagcattaaatcctgatcatcctgtaactagaggaactgcacaaaatcctgatatatattttcaagaaagagaagtaagtaatacttattatgaagcattacctgaaatagtagaaggatatatgcaagaaatgactaaattaactggaagagaatatcatttatttaattattatggagcaaaagatgcagaaagaataataatagcaatgggaagtgtatgtgaaactgtagaagaagtaatagattatttaactactaaaggagaaaaagtaggattattaactgtacatttatatagaccttttagtataaaacattttatgaaatatatacctaaaactgtaaaaaaaatagcagtattagatagaactaaagaacctggaagtataggagaacctttatatttagatgtaaaaaatgctttttatggacaagaagtacaacctgtaatagttggaggaagatatggattaggatctaaagatgtattacctagtcatatattacctgtatttgaaaatttaaaaagtgataaacctaaagatagatttactttaagtatagtagatgatgtaactaatactagtttacctgttggagaagatataaatactactcctgaaggaactactgcatgtaaattttggggattaggaagtgatggaactgttggagcaaataaatctgcaataaaaataataggagatcatactgatatgtatgcacaaggatattttgcttatgatagtaaaaaaagtggaggaataactgtaagtcatttaagatttggaaaaagtcctataaaaagtccttatttaatagataaagcagattttgtagcagtacataatcaaagttatgttcataaatatgatgtattagcaggattaaaaaaaggaggaaattttttattaaatactgtatggactcaagaagaattagaaaaagaattacctgcaagtatgaaaaagtatatagcaaataatgatataaagttttatactttaaatgcagtaaaaatagcacaagaaataggattaggaggaagaataaatatgatatgtcaaagtgcattttttaaaatagcaaatataatacctatagatgatgcagtaaaatatttaaaagaagcagtagtaactagttatggaaaaaaaggacaaaaagttgtagatatgaataatgcagcaatagataaaggagtaaatgcagtagtaaaaatagatgtacctgcaagttggaaagatgctgaagatgaagcagcagcaactaaagaattacctaaatttatagaaaaaatagtaaatcctatgaatagacaagaaggagataaattacctgtaagtgcatttgtaggaatggaagatggaacttttcctgctggaactgcagcttatgaaaaaagaggaatagcaataaatgtacctgaatggcaagtagataaatgtatacaatgtaatcaatgtagttttgtatgtcctcatgcagcaataagacctgtattaactactgaagaagaattagctaaagcacctcaaggatttgaagctaaagatgcaaatggagctaaaggattaaaatttactatggcaataagtcctttagattgtagtggatgtggaaattgtgaagatgtatgtcctgcaaaagaaaaagcattagtaatgaaacctgtagatactcaattaagtaaaactgaagcatgggattatgcagtaaatgctgtagcacataaagataatcctatgaaagataaatatagtgtaaaagcaagtcaatttgaacaacctttattagaatttagtggagcatgtgcaggatgtggagaaactccttatgtaaaattagtaactcaattatttggagatagaatgatgatagcaaatgcaactggatgtagtagtatatggggagcaagtgctcctgcaactccttatactactaattatagaggacatggacctagttgggcaaattctttatttgaagataatgcagaatatggattaggaatgtttttaggagtaaaacaaactagagaaagattacaagataaaatagaagaagcattaaaaggatctttaagtgcagaattaaaagcagcatttgaagattggataaaaaattttgcagaaggagaaggaacaagagaaagagcagataaaataactgcattattagaaaaagaaaaaggatctaatgatttattaaatgatatatatgaaaatagagattttttagtaaaaagaagtcattggataataggaggagatggatggggatatgatataggatatggaggagtagatcatgtattagcaagtaatgaagatgtaaatatattagtattagatactgaagtatatagtaatactggaggacaaagtagtaaaagtactcctactgcagcagtagcaaaatttgcagcaagtggaaaaaaaactaaaaaaaaagatttaggaatgatggcaatgagttatggatatgtatatgtagcacaaataagtatgggagctgataaaaatcaagctttaaaagcaatacatgaagctgaagcatatcatggaccaagtttaataatagcttatgcaccttgtataaatcatggattaagagtaggaatgggaaaaagtcaaagagaagcaaaaagagcagtagattgtggatattgggcattatatagatataatcctgaattaaaagaagaaggaaaaaaatcttttagtttagatagtaaagaacctactactgattttaaagaatttttaatgggagaagtaagatatagtagtttagcaaaacaatttcctgatcaagcagatgctttatttgaaaaaacaaaaaaagatgcattacaaagaatagctggatataaaaaattagataatgaacaataagctagc

SEQ ID NO: 13 - DNA, 25 bp, Artificial sequence
  Synthetic primer
aagaagggcgtatatgaaaacttgt

SEQ ID NO: 14 - DNA, 25 bp, Artificial sequence
  Synthetic primer
ttcgtttacaaaacggcaaatgtga

SEQ ID NO: 15 - DNA, 573 bp, Clostridium autoethanogenum
aagcggccgcagatagtcataatagttccagaatagttcaatttagaaattagactaaacttcaaaatgtttgttaaatatataccaaactagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcaattgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaatagggttttaggtaaaattaagttattttaagaagtaattacaataaaaattgaagttattgctttaaggagggaattattcatatggaa

SEQ ID NO: 16 - DNA, 1824 bp, Clostridium autoethanogenum
atgtatggttatgatggtaaagtattaagaattaatttaaaagaaagaacttgcaaatcagaaaatttagatttagataaagctaaaaagtttataggttgtaggggactaggtgttaaaactttatttgatgaaatagatcctaaaatagatgcattatcaccagaaaataaatttataattgtaacaggtcctttaactggagctccggttccaactagtggaaggtttatggtagttactaaagcaccgcttacaggaactataggaatttcaaattcgggtggaaaatggggagtagacttaaaaaaagctggttgggatatgataatagtagaggataaggctgattcaccagtttacattgaaatagtagatgataaggtagaaattaaagacgcgtcacagctttggggaaaagttacatcagaaactacaaaagagttagaaaagataactgagaataaatcaaaggtattatgtataggacctgctggtgaacgattgtctcttatggcagcagttatgaatgatgtagatagaactgcagcaagaggcggcgttggtgcagttatgggatctaaaaacttaaaagctattacagttaaaggaactggaaaaatagctttagctgataaagaaaaagtaaaaaaagtgtccgtagaaaaaattacaacattaaaaaatgatccagtagctggtcagggaatgccaacttatggtacagctatactggttaatataataaatgaaaatggagttcatcctgtaaagaattttcaagagtcttatacgaatcaagcagataaaataagtggagagactcttactgctaaccaactagtaaggaaaaatccttgttacagctgtcctataggttgtggaagatgggttagactaaaagatggcacagagtgcggaggaccagaatatgaaacactgtggtgttttggatctgactgtggttcatatgatttagatgctataaatgaagctaatatgttatgtaatgaatatggtattgatactattacttgtggtgcaacaattgctgcagctatggaactttatcaaagaggatatataaaagacgaagaaatagctggagataacctatctctcaagtggggtgatacggaatctatgattggctggataaagagaatggtatatagtgaaggctttggagcaaagatgacaaatggttcatataggctttgtgaaggttatggagcaccggagtattctatgacagttaaaaagcaggaaattccagcatatgatccaaggggaatacagggacacggtattacctatgcagttaataatagaggaggctgtcatattaagggatacatgattaaccctgaaatattaggttatcctgaaaaacttgatagatttgcattagatggtaaagcagcttatgccaaattatttcatgatttaactgctgtaattgattctttaggattgtgcatattcactacatttgggcttggaatacaggattatgtagatatgtataatgcagtagtaggagaatctacttatgatgcagattcactattagaggcaggagatagaatctggactcttgagaaattatttaatcttgcagctggaatagacagcagccaggatactctaccaaagagattgttagaagaacctattccagatggcccatcaaagggagaagttcataggctagatgttcttctgccagaatattactcagtacgaggatggagtaaagagggtatacctacagaagaaacattaaagaaattaggattagatgaatatataggtaagttctag

SEQ ID NO: 17 - DNA, 6832 bp, Artificial sequence
  Synthetic plasmid
cctgcaggataaaaaaattgtagataaattttataaaatagttttatctacaatttttttatcaggaaacagctatgaccgcggccgcagatagtcataatagttccagaatagttcaatttagaaattagactaaacttcaaaatgtttgttaaatatataccaaactagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcaattgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaaatagggttttaggtaaaattaagttattttaagaagtaattacaataaaaattgaagttatttctttaaggagggaattattcatatgtatggttatgatggtaaagtattaagaattaatttaaaagaaagaacttgcaaatcagaaaatttagatttagataaagctaaaaagtttataggttgtaggggactaggtgttaaaactttatttgatgaaatagatcctaaaatagatgcattatcaccagaaaataaatttataattgtaacaggtcctttaactggagctccggttccaactagtggaaggtttatggtagttactaaagcaccgcttacaggaactataggaatttcaaattcgggtggaaaatggggagtagacttaaaaaaagctggttgggatatgataatagtagaggataaggctgattcaccagtttacattgaaatagtagatgataaggtagaaattaaagacgcgtcacagctttggggaaaagttacatcagaaactacaaaagagttagaaaagataactgagaataaatcaaaggtattatgtataggacctgctggtgaacgattgtctcttatggcagcagttatgaatgatgtagatagaactgcagcaagaggcggcgttggtgcagttatgggatctaaaaacttaaaagctattacagttaaaggaactggaaaaatagctttagctgataaagaaaaagtaaaaaaagtgtccgtagaaaaaattacaacattaaaaaatgatccagtagctggtcagggaatgccaacttatggtacagctatactggttaatataataaatgaaaatggagttcatcctgtaaagaattttcaagagtcttatacgaatcaagcagataaaataagtggagagactcttactgctaaccaactagtaaggaaaaatccttgttacagctgtcctataggttgtggaagatgggttagactaaaagatggcacagagtgcggaggaccagaatatgaaacactgtggtgttttggatctgactgtggttcttatgatttagatgctataaatgaagctaatatgttatgtaatgaatatggtattgatactattacttgtggtgcaacaattgctgcagctatggaactttatcaaagaggatatataaaagacgaagaaatagctggagataacctatctctcaagtggggtgatacggaatctatgattggctggataaagagaatggtatatagtgaaggctttggagcaaagatgacaaatggttcatataggctttgtgaaggttatggagcaccggagtattctatgacagttaaaaagcaggaaattccagcttatgatccaaggggaatacagggacacggtattacctatgcagttaataatagaggaggctgtcatattaagggatacatgattaaccctgaaatattaggttatcctgaaaaacttgatagatttgcattagatggtaaagcagcttatgccaaattatttcatgatttaactgctgtaattgattctttaggattgtgcatattcactacatttgggcttggaatacaggattatgtagatatgtataatgcagtagtaggagaatctacttatgatgcagattcactattagaggcaggagatagaatctggactcttgagaaattatttaatcttgcagctggaatagacagcagccaggatactctaccaaagagattgttagaagaacctattccagatggcccatcaaagggagaagttcataggctagatgttcttctgccagaatattactcagtacgaggatggagtaaagagggtatacctacagaagaaacattaaagaaattaggattagatgaatatataggtaagttctagtttgattcgggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 18 - DNA, 29 bp, Artificial sequence
  Synthetic primer
aagcggccgcagatagtcataatagttcc

SEQ ID NO: 19 - DNA, 29 bp, Artificial sequence
  Synthetic primer
ttccatatgaataattccctccttaaagc

SEQ ID NO: 20 - DNA, 36 bp, Artificial sequence
  Synthetic primer
aattcatatgtatggttatgatggtaaagtattaag

SEQ ID NO: 21 - DNA, 28 bp, Artificial sequence
  Synthetic primer
ctaaatcataagaaccacagtcagatcc

SEQ ID NO: 22 - DNA, 28 bp, Artificial sequence
  Synthetic primer
ctgactgtggttcttatgatttagatgc

SEQ ID NO: 23 - DNA, 25 bp, Artificial sequence
  Synthetic primer
cctgtattccccttggatcataagc

SEQ ID NO: 24 - DNA, 25 bp, Artificial sequence
  Synthetic primer
gcttatgatccaaggggaatacagg

SEQ ID NO: 25 - DNA, 30 bp, Artificial sequence
  Synthetic primer
ctagaattccgaatcaaactagaacttacc

SEQ ID NO: 26 - DNA, 18 bp, Artificial sequence
  Synthetic primer
tgtaaaacgacggccagt

SEQ ID NO: 27 - DNA, 18 bp, Artificial sequence
  Synthetic primer
caggaaacagctatgacc

SEQ ID NO: 28 - DNA, 33 bp, Artificial sequence
  Synthetic primer
atgtggacaaagttacaaaagttcttgaggaac

SEQ ID NO: 29 - DNA, 37 bp, Artificial sequence
  Synthetic primer
gtaaatattcaaatatcaactttactgcttcaagggc

(19) United States
(12) Patent Application Publication
(10) Pub. No.: US 2015/0079650 A1
(43) Pub. Date: Mar. 19, 2015

(54) Recombinant microorganisms exhibiting increased flux through a fermentation pathway

(71) Applicant: LanzaTech New Zealand Limited, Auckland (NZ)

(72) Inventors: Michael Koepke, Auckland (NZ); Alexander Paul Mueller, Auckland (NZ)

(21) Appl. No.: 14/297,577

(22) Filed: Jun. 5, 2014

Related U.S. Application Data
(60) Provisional application No. 61/831,591, filed on Jun. 5, 2013.

(51) Int. Cl. C12P 7/06; C12N 9/02; C12N 9/10; C12N 9/04; C12P 7/18; C12N 15/74

(52) U.S. Cl. CPC C12P 7/06; C12P 7/18; C12N 15/74; C12N 9/1007; C12N 9/0006; C12N 9/0008; C12Y 101/01001; C12Y 102/0101; C12Y 102/07001; C12Y 102/07005; C12Y 201/01196
USPC 435/158; 435/161; 435/471; 435/252.3

(57) ABSTRACT

The invention provides methods of increasing the production of fermentation products by increasing flux through a fermentation pathway by optimising enzymatic reactions. In particular, the invention relates to identifying enzymes and/or co-factors involved in metabolic bottlenecks in fermentation pathways, and fermenting a CO-comprising substrate with a recombinant carboxydotrophic Clostridia microorganism adapted to exhibit increased activity of the one or more of said enzymes, or increased availability of the one or more of said co-factors, when compared to a parental microorganism.

### CROSS-REFERENCE TO RELATED APPLICATION

This application claims priority from Provisional Application No. 61/831,591 filed Jun. 5, 2013, the contents of which are hereby incorporated by reference.

### FIELD OF INVENTION

The invention relates to methods of increasing flux through a fermentation pathway by optimising enzymatic reactions. More particularly, the invention relates to identifying and addressing reaction bottlenecks in fermentation pathways.

### BACKGROUND

Acetogenic microorganisms are known to be useful for the production of fuels and other chemicals (for example, ethanol, butanol or butanediol) by fermentation of substrates including carbon monoxide, carbon dioxide and hydrogen, for example.

Efforts so far to improve product concentration and substrate utilization have focused on strain selection, optimisation of fermentation conditions and parameters as well as optimisation of media formulation or process conditions (Abubackar et al., 2012). The metabolism of natural organisms, however, did not evolve to achieve commercial objectives of high yields, rates and titers (Nielsen, 2011). While the rate of certain reactions in the organism can be increased by optimization of process conditions or strain selection, there are typically some reactions that are not affected and will be rate limiting.

In the last decade, a number of in silico predictive tools have been developed to explore genome scale metabolic reconstructions. These constraint-based models use a stoichiometric approach to study the flux through metabolic networks, in which all possible net flux distributions (feasible flux space) are constrained by observed cellular input and output measurements (external fluxes) and by mass balance and thermodynamic equations. Flux balance analysis (FBA) probes this solution space to identify metabolic flux distributions that optimize certain objectives (usually maximizing growth). Flux balance analysis requires very little information in terms of the enzyme kinetic parameters and concentration of metabolites in the system. However, one limitation of such an approach is that it is unable to identify rate limiting reactions (bottlenecks).

It is an object of the invention to provide a method of increasing the efficiency of a fermentation reaction, or at least to provide the public with a useful choice.

### SUMMARY OF THE INVENTION

In a first aspect, the invention provides a method of producing a fermentation product, the method comprising at least the steps of:
a) determining a rate-limiting pathway reaction in a fermentation pathway;
b) identifying one or more enzymes, co-factors or both, which are involved in catalysing the rate-limiting pathway reaction;
c) fermenting a CO-comprising substrate with a recombinant carboxydotrophic Clostridia microorganism adapted to exhibit at least one of: i) increased activity of the one or more enzymes of b) or a functionally equivalent variant of any one or more thereof, or ii) increased availability of the one or more co-factors of b), when compared to a parental microorganism, to produce a fermentation product.

In a second aspect, the invention provides a method of increasing the flux through a fermentation pathway, the method comprising at least the steps of:
a) determining a rate-limiting pathway reaction in the fermentation pathway;
b) identifying one or more enzymes, co-factors or both, involved in catalysing the rate-limiting pathway reaction;
c) fermenting a CO-comprising substrate with a recombinant carboxydotrophic Clostridia microorganism adapted to exhibit at least one of i) increased activity of the one or more enzymes of b) or a functionally equivalent variant of any one or more thereof, or ii) increased availability of the one or more co-factors of b), when compared to a parental microorganism.

In a third aspect, the invention provides a method of producing a recombinant carboxydotrophic Clostridia microorganism adapted to exhibit increased flux through a fermentation pathway relative to a parental microorganism, the method comprising:
a) determining a rate limiting pathway reaction in the fermentation pathway;
b) identifying one or more enzymes, co-factors or both, which are involved in catalysing the rate-limiting pathway reaction;
c) transforming a parental microorganism to yield a recombinant microorganism adapted to exhibit at least one of i) increased activity of the one or more enzymes of b) or a functionally equivalent variant of any one or more thereof, or ii) increased availability of the one or more co-factors of b), when compared to a parental microorganism;
   wherein the fermentation pathway is capable of producing one or more fermentation products from a substrate comprising CO.

In a fourth aspect, the invention provides a method of producing a fermentation product, the method comprising fermenting a CO-comprising substrate with a recombinant carboxydotrophic Clostridia microorganism to produce a fermentation product, wherein the recombinant microorganism is adapted to exhibit at least one of:
i) increased activity of one or more enzymes identified as being involved in catalysing a rate-limiting pathway reaction of a fermentation pathway, or a functionally equivalent variant of any one or more thereof, when compared to a parental microorganism; or
ii) increased availability of one or more co-factors identified as being involved in catalysing a rate-limiting pathway reaction of a fermentation pathway, when compared to a parental microorganism.

In a particular embodiment of any one of the preceding aspects, the recombinant microorganism is adapted to:
i) over-express the one or more enzymes identified as being involved in catalysing a rate-limiting pathway reaction or a functionally equivalent variant of any one or more thereof; or
ii) express one or more exogenous enzymes identified as being involved in catalysing a rate-limiting pathway reaction; or
iii) both i) and ii).

In a particular embodiment of any one of the preceding aspects, the recombinant microorganism has undergone enzyme engineering to increase the activity of the enzyme or increase the availability of the one or more co-factor identified as being involved in catalysing a rate-limiting pathway reaction. In a particular embodiment, the method of enzyme engineering is selected from the group consisting of directed evolution, knowledge based design, random mutagenesis methods, gene shuffling, codon optimization, use of site-specific libraries and use of site evaluation libraries.

In a particular embodiment of any one of the preceding aspects, the recombinant microorganism is adapted to exhibit an increase in efficiency of the fermentation pathway relative to the parental microorganism. Preferably, the increase in efficiency comprises an increase in the rate of production of a fermentation product.

In a particular embodiment of any one of the preceding aspects, the rate-limiting pathway reaction is determined by analysis of the enzymatic activity of two or more pathway reactions that make up the fermentation pathway.

In a particular embodiment of any one of the preceding aspects, the rate-limiting pathway reaction is the pathway reaction with the lowest enzymatic activity.

In a particular embodiment of the first, third or fourth aspects, the one or more fermentation products are at least one of ethanol, butanol, isopropanol, isobutanol, higher alcohols, butanediol, 2,3-butanediol, succinate, isoprenoids, fatty acids, or biopolymers.

In a particular embodiment of any one of the preceding aspects, the fermentation pathway is the Wood-Ljungdahl, ethanol or 2,3-butanediol fermentation pathway.

In a particular embodiment of any one of the preceding aspects, the one or more enzymes are selected from the group consisting of alcohol dehydrogenase (EC 1.1.1.1), aldehyde dehydrogenase (acylating) (EC 1.2.1.10), formate dehydrogenase (EC 1.2.1.2), formyl-THF synthetase (EC 6.3.2.17), methylene-THF dehydrogenase/formyl-THF cyclohydrolase (EC:6.3.4.3), methylene-THF reductase (EC 1.1, 1.58), CO dehydrogenase/acetyl-CoA synthase (EC 2.3.1.169), aldehyde ferredoxin oxidoreductase (EC 1.2.7.5), phosphotransacetylase (EC 2.3.1.8), acetate kinase (EC 2.7.2.1), CO dehydrogenase (EC 1.2.99.2), and hydrogenase (EC 1.12.7.2). Preferably, the microorganism of this embodiment is adapted to exhibit an increase in the flux through a fermentation pathway resulting in the production of ethanol.

In a particular embodiment of any one of the preceding aspects, the one or more enzymes is selected from the group consisting of pyruvate:ferredoxin oxidoreductase (Pyruvate synthase) (EC 1.2.7.1), pyruvate:formate lyase (EC 2.3.1.54), acetolactate synthase (EC 2.2.1.6), acetolactate decarboxylase (EC 4.1.1.5), 2,3-butanediol dehydrogenase (EC 1.1.1.4), primary:seconday alcohol dehydrogenase (EC 1.1.1.1), formate dehydrogenase (EC 1.2.1.2), formyl-THF synthetase (EC 6.3.2.17), methylene-THF dehydrogenase/formyl-THF cyclohydrolase (EC:6.3.4.3), methylene-THF reductase (EC 1.1, 1.58), CO dehydrogenase/acetyl-CoA synthase (EC 2.3.1.169), CO dehydrogenase (EC 1.2.99.2), and hydrogenase (EC 1.12.7.2). Preferably, the microorganism of this embodiment is adapted to exhibit an increase in the flux through a fermentation pathway resulting in the production of 2,3-butanediol.

In a particular embodiment of any one of the preceding aspects, the recombinant microorganism is adapted to express an exogenous nucleic acid, or over-express an endogenous nucleic acid involved in the biosynthesis of an enzyme or co-factor involved in catalysing the rate limiting pathway reaction. In a particular embodiment, the endogenous or exogenous nucleic acid encodes an enzyme selected from the enzymes above.

In a particular embodiment of any one of the preceding aspects, the recombinant microorganism is adapted to exhibit increased availability of one or more co-factors. The increase in availability may come about as a result of altered expression of an endogenous nucleic acid, or expression of an exogenous nucleic acid, wherein the endogenous or exogenous nucleic acid is involved in the biosynthesis of a co-factor involved in catalysing the rate limiting pathway reaction.

In a particular embodiment, the co-factor comprises tetrahydrofolate (THF). In a particular embodiment, the recombinant microorganism exhibits increased expression of at least one of GTP cyclohydrolase I (EC 3.5.4.16), alkaline phosphatase (EC 3.1.3.1), dihydroneopterin aldolase (EC 4.1.2.25), 2-amino-4-hydroxy-6-hydroxymethyldihydropteridine diphosphokinase (EC 2.7.6.3), dihydropteroate synthase (2.5.1.15), dihydropteroate synthase (EC 2.5.1.15), dihydrofolate synthase (EC 6.3.2.12), folylpolyglutamate synthase (6.3.2.17), dihydrofolate reductase (EC 1.5.1.3), thymidylate synthase (EC 2.1.1.45), or dihydromonapterin reductase (EC 1.5.1.-).

In a particular embodiment, the co-factor comprises cobalamine (B₁₂). In a particular embodiment, the recombinant microorganism exhibits increased expression of at least one of 5-aminolevulinate synthase (EC 2.3.1.37), 5-aminolevulinate:pyruvate aminotransferase (EC 2.6.1.43), adenosylcobinamide kinase/adenosylcobinamide-phosphate guanylyltransferase (EC 2.7.1.156/2.7.7.62), adenosylcobinamide-GDP ribazoletransferase (EC 2.7.8.26), adenosylcobinamide-phosphate synthase (EC 6.3.1.10), adenosylcobyric acid synthase (EC 6.3.5.10), alpha-ribazole phosphatase (EC 3.1.3.73), cob(I)alamin adenosyltransferase (EC 2.5.1.17), cob(II)yrinic acid a,c-diamide reductase (EC 1.16.8.1), cobalt-precorrin 5A hydrolase (EC 3.7.1.12), cobalt-precorrin-5B (C1)-methyltransferase (EC 2.1.1.195), cobalt-precorrin-7 (C15)-methyltransferase (EC 2.1.1.196), cobaltochelatase CobN (EC 6.6.1.2), cobyrinic acid a,c-diamide synthase (EC 6.3.5.9/6.3.5.11), ferritin (EC 1.16.3.1), glutamate-1-semialdehyde 2,1-aminomutase (EC 5.4.3.8), glutamyl-tRNA reductase (EC 1.2.1.70), glutamyl-tRNA synthetase (EC 6.1.1.17), hydroxymethylbilane synthase (EC 2.5.1.61), nicotinate-nucleotide-dimethylbenzimidazole phosphoribosyltransferase (EC 2.4.2.21), oxygen-independent coproporphyrinogen III oxidase (EC 1.3.99.22), porphobilinogen synthase (EC 4.2.1.24), precorrin-2 dehydrogenase/sirohydrochlorin ferrochelatase (EC 1.3.1.76/4.99.1.4), precorrin-2/cobalt-factor-2 C20-methyltransferase (EC 2.1.1.130/2.1.1.151), precorrin-3B synthase (EC 1.14.13.83), precorrin-3B C17-methyltransferase (EC 2.1.1.131), precorrin-4 C11-methyltransferase (EC 2.1.1.133), precorrin-6X reductase (EC 1.3.1.54), precorrin-6Y C5,15-methyltransferase (EC 2.1.1.132), precorrin-8W decarboxylase (EC 1.-.-.-), precorrin-8X methylmutase (EC 5.4.1.2), sirohydrochlorin cobaltochelatase (EC 4.99.1.3), threonine-phosphate decarboxylase (EC 4.1.1.81), uroporphyrinogen decarboxylase (EC 4.1.1.37), or uroporphyrinogen III methyltransferase/synthase (EC 2.1.1.107/4.2.1.75).

In a fifth aspect, the invention provides a recombinant carboxydotrophic Clostridia microorganism produced by the method of the third aspect.

In a sixth aspect, the invention provides a recombinant carboxydotrophic Clostridia microorganism adapted to exhibit at least one of:
a) increased activity of one or more enzymes or a functionally equivalent variant of any one or more thereof when compared to a parental microorganism;
b) increased availability of one or more co-factors when compared to a parental microorganism; or
c) both a) and b);
   wherein the enzymes or co-factors have been identified as being involved in catalysing a rate-limiting pathway reaction.

In a seventh aspect, the invention provides the use of a microorganism according to the fifth or sixth aspect to increase the flux through a reaction pathway.

In an eighth aspect, the invention provides a method of producing a fermentation product, the method comprising at least the steps of:
a) determining a rate-limiting pathway reaction in the Wood-Ljungdahl, ethanol or 2,3-butanediol fermentation pathways;
b) identifying one or more enzymes, co-factors or both, which are involved in catalysing the rate-limiting pathway reaction;
c) fermenting a CO-comprising substrate with a recombinant carboxydotrophic Clostridia microorganism adapted to express or over-express a gene or a functionally equivalent variant thereof which encodes the one or more enzymes or co-factors of b) when compared to a parental microorganism, to produce a fermentation product.

In a particular embodiment, the enzyme of the eighth aspect is AOR1 and the fermentation product is ethanol.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE DRAWINGS

Embodiments of the invention will now be described, by way of example only, with reference to the accompanying drawings in which:

FIG. 1 shows a flux map of the ethanol biosynthesis pathway detailing the measured enzyme activities and flux through the carboxydotrophic cell for ethanol formation via acetyl-CoA which allows the identification of rate-limiting pathway reactions. The thickness of the arrows is proportional to the activity of the particular pathway reaction; and

FIG. 2 shows a flux map detailing the measured enzyme activities and flux through the carboxydotrophic cell for 2,3-butanediol formation via pyruvate which allows the identification of rate-limiting pathway reactions.

FIG. 3 shows a sequence alignment of the insert and promoter of the expression plasmid pMTL83157-AOR1AOR1 confirming that two internal NdeI sites of AOR1 were successfully altered and they were free of mutations.

FIG. 4 shows the presence of the expected 576 bp product in both plasmid control and AOR1 overexpression strains illustrating successful transformation to produce a recombinant microorganism.

FIG. 5 shows the presence of the expected fragments following NdeI and KpnI digestions of rescued plasmids from pMTL83157-AOR1 transformants.

FIG. 6 shows that the overexpression of AOR1 (crosses, upper line at day 10) improves autotrophic growth of C. autoethanogenum DSM10061 relative to plasmid control.

FIG. 7A shows ethanol production in C. autoethanogenum wild-type strain (crosses, lower line at day 10) versus C. autoethanogenum recombinant strain with AOR1 overexpression (squares, upper line at day 10).

FIG. 7B shows acetate production in C. autoethanogenum wild-type strain (crosses, lower line at day 10) versus C. autoethanogenum recombinant strain with AOR1 overexpression (squares, upper line at day 10).

### DETAILED DESCRIPTION OF THE INVENTION

### Definitions

As referred to herein, a “fermentation pathway” is a cascade of biochemical reactions (referred to herein as “pathway reactions”) by which a substrate, preferably a gaseous substrate, is converted to a fermentation product. The pathway reactions typically involve enzymes and may involve co-factors whereby the enzyme or co-factor facilitates or increases the rate of the pathway reaction.

As referred to herein, a “rate-limiting pathway reaction” is a reaction which is part of a fermentation pathway, and is a “bottleneck” in the pathway whereby flux through the entire pathway is slowed and determined by the rate of reaction of the rate-limiting pathway reaction. With all other factors being constant, increasing the rate of reaction of the rate limiting pathway reaction has a knock-on effect on the rate of the overall fermentation pathway and potentially the production of the one or more fermentation products. Where “a” or “the” (singular) rate-limiting pathway reaction is referred to herein, it should be understood that multiple (for example 2 or more) rate-limiting pathway reactions are also included within the scope of the invention and such multiple reactions may also be determined and altered according to the methods described herein.

As referred to herein, reaction “flux” refers to the flow of metabolites through one or more reactions in a fermentation pathway. The flux through individual pathway reactions has an upper and lower limit therefore the flux may be changed by the adjustment of conditions or factors that affect enzymatic activity. Adjustment of the flux through one pathway reaction may alter the overall flux of the fermentation pathway. Flux may be measured according to methods known to one of skill in the art. By way of example, flux may be measured using flux-balance analysis (FBA) (Gianchandani et al., 2010). In a particular embodiment, the flux is increased by at least 5%, at least 10%, at least 20%, at least 30%, at least 50%, at least 100%. Flux through the pathway may also be measured by the level of metabolites and products (metabolomics) (Patti et al., 2012) and/or labelling experiments as C13 (fluxomics) (Niittylae et al., 2009; Tang et al., 2009).

The term “nicotinamide adenine dinucleotide” (NADH) refers to either NAD+ (oxidized form), NADH+H+ (reduced form) or the redox couple of both NAD+ and NADH+H+.

The term “nicotinamide adenine dinucleotide phosphate” (NADPH) refers to either NADP+ (oxidized form), NADPH+H+ (reduced form) or the redox couple of both NADP+ and NADPH+H+.

As referred to herein, an “enzyme co-factor”, or simply a “co-factor” is a non-protein compound that binds to an enzyme to facilitate the biological function of the enzyme and thus the catalysis of a reaction. Non-limiting examples of co-factors include NAD+, NADP+, cobalamine, tetrahydrofolate and ferredoxin. Increase in the overall availability of the co-factor can increase the rate of a pathway reaction. Factors that may affect production of the co-factor include the expression of co-factor biosynthesis genes which may be altered to achieve increased availability of the co-factor. Other factors known to one of skill in the art may also be used to achieve increased availability of the co-factor. Lack of availability of co-factors can have rate-limiting effects on pathway reactions. Methods for the determination of availability of co-factors will be known to those of skill in the art.

The term “adapted to” may be used herein to describe the function of a recombinant microorganism of the invention; for example, the microorganism is “adapted to” express a particular enzyme. When used in relation to the expression of an enzyme, the term does not imply that the enzyme is continuously expressed, it is intended to cover situations where the enzyme may be expressed and such expression may be constitutive or induced.

As referred to herein, a “fermentation broth” is a culture medium comprising at least nutrients and microorganism cells.

The terms “increasing the efficiency”, “increased efficiency” and the like, when used in relation to a fermentation pathway or process, include, but are not limited to at least one of: an increased rate of growth of microorganisms effecting the fermentation; an increased rate of growth or product production rate at elevated product concentrations; an increased fermentation product concentration in the fermentation broth; an increased volume of fermentation product produced per volume of substrate consumed; an increased rate of production or level of production of the fermentation product. The increases in efficiency are measured relative to the corresponding variable as measured when using a parental microorganism.

“Enzyme activity”, “activity of one or more enzymes” and like phrases should be taken broadly to refer to enzymatic activity, including but not limited to the activity of an individual enzyme, the amount of enzyme, or the availability of an enzyme. Accordingly, where reference is made to “increasing” enzyme activity, it should be taken to include an increase in the activity of an individual enzyme, an increase in the amount of the enzyme, or an increase in the availability of an enzyme to catalyse a particular reaction.

The phrase “Involved in catalysing” is intended to encompass enzymes which directly catalyse (i.e. facilitate or increase the rate of) a reaction, as well as co-factors which do not directly catalyse a reaction but facilitate the biological function of an associated enzyme.

The phrase “substrate comprising carbon monoxide” and like terms should be understood to include any substrate in which carbon monoxide is available to one or more strains of bacteria for growth and/or fermentation, for example.

The phrase “gaseous substrate comprising carbon monoxide” and like phrases and terms includes any gas which contains a level of carbon monoxide. In certain embodiments the substrate contains at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

While it is not necessary for the substrate to contain any hydrogen, the presence of H₂ should not be detrimental to product formation in accordance with methods of the invention. In particular embodiments, the presence of hydrogen results in an improved overall efficiency of alcohol production. For example, in particular embodiments, the substrate may comprise an approx. 2:1, or 1:1, or 1:2 ratio of H₂:CO. In one embodiment the substrate comprises about 30% or less H₂ by volume, 20% or less H₂ by volume, about 15% or less H₂ by volume or about 10% or less H₂ by volume. In other embodiments, the substrate stream comprises low concentrations of H₂, for example, less than 5%, or less than 4%, or less than 3%, or less than 2%, or less than 1%, or is substantially hydrogen free. The substrate may also contain some CO₂ for example, such as about 1% to about 80% CO₂ by volume, or 1% to about 30% CO₂ by volume. In one embodiment the substrate comprises less than or equal to about 20% CO₂ by volume. In particular embodiments the substrate comprises less than or equal to about 15% CO₂ by volume, less than or equal to about 10% CO₂ by volume, less than or equal to about 5% CO₂ by volume or substantially no CO₂.

In the description which follows, embodiments of the invention are described in terms of delivering and fermenting a “gaseous substrate containing CO”. However, it should be appreciated that the gaseous substrate may be provided in alternative forms. For example, the gaseous substrate containing CO may be provided dissolved in a liquid. Essentially, a liquid is saturated with a carbon monoxide containing gas and then that liquid is added to the bioreactor. This may be achieved using standard methodology. By way of example, a microbubble dispersion generator (Hensirisak et. al. Scale-up of microbubble dispersion generator for aerobic fermentation; Applied Biochemistry and Biotechnology Volume 101, Number 3/October, 2002) could be used. By way of further example, the gaseous substrate containing CO may be adsorbed onto a solid support. Such alternative methods are encompassed by use of the term “substrate containing CO” and the like.

In particular embodiments of the invention, the CO-containing gaseous substrate is an industrial off or waste gas. “Industrial waste or off gases” should be taken broadly to include any gases comprising CO produced by an industrial process and include gases produced as a result of ferrous metal products manufacturing, non-ferrous products manufacturing, petroleum refining processes, gasification of coal, gasification of biomass, electric power production, carbon black production, and coke manufacturing. Further examples may be provided elsewhere herein.

Unless the context requires otherwise, the phrases “fermenting”, “fermentation process” or “fermentation reaction” and the like, as used herein, are intended to encompass both the growth phase and product biosynthesis phase of the process. As will be described further herein, in some embodiments the bioreactor may comprise a first growth reactor and a second fermentation reactor. As such, the addition of metals or compositions to a fermentation reaction should be understood to include addition to either or both of these reactors.

The term “bioreactor” includes a fermentation device consisting of one or more vessels and/or towers or piping arrangement, which includes the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. In some embodiments the bioreactor may comprise a first growth reactor and a second fermentation reactor. As such, when referring to the addition of substrate to the bioreactor or fermentation reaction it should be understood to include addition to either or both of these reactors where appropriate.

As referred to herein, a “shuttle microorganism” is a microorganism in which a methyltransferase enzyme is expressed and is distinct from the destination microorganism.

As referred to herein, a “destination microorganism” is a microorganism in which the genes included on an expression construct/vector are expressed and is distinct from the shuttle microorganism.

“Exogenous nucleic acids” are nucleic acids which originate outside of the microorganism to which they are introduced. Exogenous nucleic acids may be derived from any appropriate source, including, but not limited to, the microorganism to which they are to be introduced (for example in a parental microorganism from which the recombinant microorganism is derived), strains or species of microorganisms which differ from the organism to which they are to be introduced, or they may be artificially or recombinantly created. In one embodiment, the exogenous nucleic acids represent nucleic acid sequences naturally present within the microorganism to which they are to be introduced, and they are introduced to increase expression of or over-express a particular gene (for example, by increasing the copy number of the sequence (for example a gene), or introducing a strong or constitutive promoter to increase expression). In another embodiment, the exogenous nucleic acids represent nucleic acid sequences not naturally present within the microorganism to which they are to be introduced and allow for the expression of a product not naturally present within the microorganism or increased expression of a gene native to the microorganism (for example in the case of introduction of a regulatory element such as a promoter). The exogenous nucleic acid may be adapted to integrate into the genome of the microorganism to which it is to be introduced or to remain in an extra-chromosomal state.

“Exogenous” may also be used to refer to proteins. This refers to a protein that is not present in the parental microorganism from which the recombinant microorganism is derived.

The term “endogenous” as used herein in relation to a recombinant microorganism and a nucleic acid or protein refers to any nucleic acid or protein that is present in a parental microorganism from which the recombinant microorganism is derived.

Oxidoreductases (or dehydrogenases, oxidases) include enzymes that catalyse the transfer of electrons from one molecule—the reductant, also called the electron donor, to another molecule—the oxidant, also called the electron acceptor. Oxidoreductases are classified as EC 1 in the EC number classification of enzymes.

It should be appreciated that the invention may be practised using nucleic acids whose sequence varies from the sequences specifically exemplified herein provided they perform substantially the same function. For nucleic acid sequences that encode a protein or peptide this means that the encoded protein or peptide has substantially the same function. For nucleic acid sequences that represent promoter sequences, the variant sequence will have the ability to promote expression of one or more genes. Such nucleic acids may be referred to herein as “functionally equivalent variants”. By way of example, functionally equivalent variants of a nucleic acid include allelic variants, fragments of a gene, genes which include mutations (deletion, insertion, nucleotide substitutions and the like) and/or polymorphisms and the like. Homologous genes from other microorganisms may also be considered as examples of functionally equivalent variants of the sequences specifically exemplified herein. These include homologous genes in species such as Clostridium acetobutylicum, Clostridium beijerinckii, C. ljungdahlii details of which are publicly available on websites such as Genbank or NCBI. The phrase “functionally equivalent variants” should also be taken to include nucleic acids whose sequence varies as a result of codon optimisation for a particular organism. “Functionally equivalent variants” of a nucleic acid herein will preferably have at least approximately 70%, preferably approximately 80%, more preferably approximately 85%, preferably approximately 90%, preferably approximately 95% or greater nucleic acid sequence identity with the nucleic acid identified.

It should also be appreciated that the invention may be practised using polypeptides whose sequence varies from the amino acid sequence of the polypeptide specifically referred to or exemplified herein. These variants may be referred to herein as “functionally equivalent variants”. A functionally equivalent variant of a protein or a peptide includes those proteins or peptides that share at least 40%, preferably 50%, preferably 60%, preferably 70%, preferably 75%, preferably 80%, preferably 85%, preferably 90%, preferably 95% or greater amino acid identity with the protein or peptide identified and has substantially the same function as the peptide or protein of interest. Such variants include within their scope fragments of a protein or peptide wherein the fragment comprises a truncated form of the polypeptide wherein deletions may be from 1 to 5, to 10, to 15, to 20, to 25 amino acids, and may extend from residue 1 through 25 at either terminus of the polypeptide, and wherein deletions may be of any length within the region; or may be at an internal location. Functionally equivalent variants of the specific polypeptides herein should also be taken to include polypeptides expressed by homologous genes in other species of bacteria, for example as exemplified in the previous paragraph.

“Substantially the same function” as used herein is intended to mean that the nucleic acid or polypeptide is able to perform the function of the nucleic acid or polypeptide of which it is a variant. For example, a variant of an enzyme of the invention will be able to catalyse the same reaction as that enzyme. However, it should not be taken to mean that the variant has the same level of activity as the polypeptide or nucleic acid of which it is a variant.

One may assess whether a functionally equivalent variant has substantially the same function as the nucleic acid or polypeptide of which it is a variant using methods known to one of skill in the art. However, by way of example, assays to test for hydrogenase, formate dehydrogenase or methylene-THF-dehydrogenase activity are described in Huang et al (2012).

“Over-express”, “over expression” and like terms and phrases when used in relation to the invention should be taken broadly to include any increase in expression of one or more proteins (including expression of one or more nucleic acids encoding same) as compared to the expression level of the protein (including nucleic acids) of a parental microorganism under the same conditions. It should not be taken to mean that the protein (or nucleic acid) is expressed at any particular level.

A “recombinant microorganism” is a microorganism that has undergone intentional genetic modification when compared to a parental microorganism. A “genetic modification” should be taken broadly and includes insertion, deletion or substitution of nucleic acids, for example.

A “parental microorganism” is a microorganism used to generate a recombinant microorganism of the invention. The parental microorganism may be one that occurs in nature (i.e. a wild type microorganism) or one that has been previously modified (i.e. it is a recombinant microorganism). The recombinant microorganisms of the invention may be modified to express or over-express one or more enzymes that were not expressed or over-expressed to a desired level in the parental microorganism, or may be modified to exhibit increased availability of one or more co-factors.

The terms nucleic acid “constructs” or “vectors” and like terms should be taken broadly to include any nucleic acid (including DNA and RNA) suitable for use as a vehicle to transfer genetic material into a cell. The terms should be taken to include plasmids, viruses (including bacteriophage), cosmids and artificial chromosomes. Constructs or vectors may include one or more regulatory elements, an origin of replication, a multicloning site and/or a selectable marker. In one particular embodiment, the constructs or vectors are adapted to allow expression of one or more genes encoded by the construct or vector. Nucleic acid constructs or vectors include naked nucleic acids as well as nucleic acids formulated with one or more agents to facilitate delivery to a cell (for example, liposome-conjugated nucleic acid, an organism in which the nucleic acid is contained). The vectors may be used for cloning or expression of nucleic acids and for transformation of microorganisms to produce recombinant microorganisms.

The efficiency of a fermentation pathway can be increased by increasing the reaction flux through the pathway. The increased flux results in one or more of: an increased rate of growth of microorganisms effecting the fermentation; an increased rate of growth and/or product production rate at elevated product concentrations; an increased fermentation product concentration in the fermentation broth; an increased volume of fermentation product produced per volume of substrate consumed; an increased rate of production or level of production of the fermentation product. Preferably, the increased efficiency results in an increased fermentation product production rate.

The inventors have demonstrated methods to identify rate limiting pathway reactions in the fermentation pathway where particular pathway reactions affect the flux through the entire pathway. In some circumstances, these rate-limiting pathway reactions are not performing to their capacity and it may be desirable to increase their individual rate. The invention as described herein enables rate limiting pathway reactions (i.e. bottlenecks) in the fermentation pathway to be identified and strategies employed to increase the activity of enzymes and/or the availability of co-factors which are involved in the rate limiting pathway reactions. This invention therefore contributes to identifying the bottleneck and adjusting the rate of the rate-limiting pathway reaction to have a concomitant effect on reaction flux through the pathway. This is the first time that rate limiting pathway reactions have been identified and addressed using recombinant carboxydotrophic microorganisms.

One method to identify rate limiting reactions (bottlenecks) is to measure enzyme activities for all reactions involved in the fermentation pathway from substrate to product. This can be done by analysing the enzymatic activity of reactions in cells growing under process conditions to identify the reactions with the lowest rates. These can then be adjusted so as not to be rate limiting thus increasing the flux throughout the system. This kind of pathway analysis and bottleneck removal has never been carried out in Clostridia species.

The methods and recombinant microorganisms described herein enable further biochemical pathways to be explored and desirable fermentation products to be produced. The methods have particular utility for pathways where the product yield in the parental microorganism may have lacked the product yield to be a viable target or the yield was so low as to be undetectable.

The invention provides a method of producing a fermentation product, the method comprising at least the steps of:
a) determining a rate-limiting pathway reaction in a fermentation pathway;
b) identifying one or more enzymes, co-factors or both, which are involved in catalysing the rate-limiting pathway reaction;
c) fermenting a CO-comprising substrate with a recombinant carboxydotrophic Clostridia microorganism adapted to exhibit at least one of: i) increased activity of the one or more enzymes of b) or a functionally equivalent variant of any one or more thereof, or ii) increased availability of the one or more co-factors of b), when compared to a parental microorganism, to produce a fermentation product.

The invention also provides a method of increasing the flux through a fermentation pathway, the method comprising at least the steps of:
a) determining a rate-limiting pathway reaction in the fermentation pathway;
b) identifying one or more enzymes, co-factors or both, involved in catalysing the rate-limiting pathway reaction;
c) fermenting a CO-comprising substrate with a recombinant carboxydotrophic Clostridia microorganism adapted to exhibit at least one of i) increased activity of the one or more enzymes of b) or a functionally equivalent variant of any one or more thereof, or ii) increased availability of the one or more co-factors of b), when compared to a parental microorganism.

The inventors have analysed the activity of enzymes involved in fermentation pathways and found that some pathway reactions exhibit substantially lower enzymatic activity than other reactions in the same pathway. This indicates that the pathway reaction is limiting the overall flux through the fermentation pathway and provides a method to identify a rate-limiting pathway reaction.

Enzymatic activity may be measured by methods known to one of skill in the art. In a particular embodiment, the enzymatic activity is measured by the method described in Huang et al (2012) and is referred to in Example 1.

Examples of fermentation pathways that are amenable to analysis of enzyme activity include the Wood-Ljungdahl pathway, fermentation pathways to produce ethanol, 2,3-butanediol or a precursor thereof such as acetyl-CoA and pyruvate, and biosynthesis pathways for cofactors tetrahydrofolate and Cobalamine (B₁₂) which may be required in fermentation pathways.

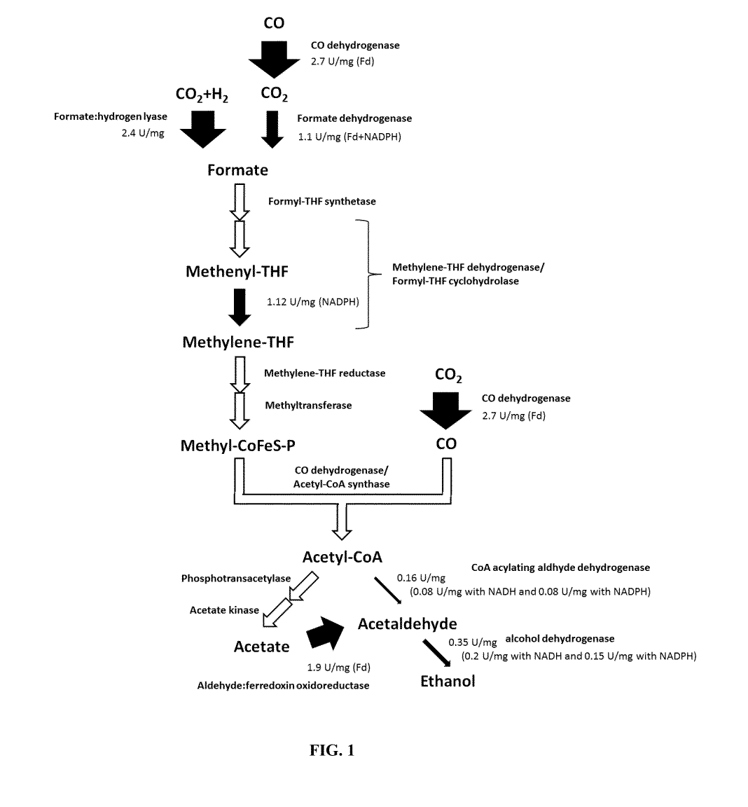

The Wood-Ljungdahl pathway is composed of a number of reactions catalysed by enzymes as described in FIGS. 1 and 2. The steps subsequent to the Wood-Ljungdahl pathway which lead to the production of desirable fermentation products are also considered to be part of the fermentation pathway.

In a particular embodiment, the fermentation pathway results in the production of a fermentation product selected from the group consisting of ethanol, butanol, acetone, isopropanol, isobutanol, 2,3-butanediol, succinate, isoprenoids, fatty acids, and biopolymers.

In one embodiment, in order to determine whether one or more of the pathway reactions is limiting the rate of flux through the fermentation pathway, the enzymatic activity of enzymes that catalyse at least two or more individual pathway reactions is compared. If, on comparison, it is found that one or more enzymes exhibit less activity than other enzymes in the same reaction pathway, this indicates that the reaction is not performing to capacity. In a particular embodiment, the activity of the enzyme is 5%, 10% or 20% less than the activity of other enzymes in the pathway. In a particular embodiment, the activity is 69% less. In another embodiment the activity is 86% less. In another embodiment the activity is 90% less, or the difference in activity is greater than 90%.

As such, in a particular embodiment, the rate-limiting pathway reaction is determined by analysis of the enzymatic activity of two or more pathway reactions that make up the fermentation pathway then designating the enzyme with the lower/lowest activity as the rate-limiting pathway reaction.

The lack of enzymatic activity may be caused by a number of factors including: lack of free enzyme to catalyse the reaction; inhibition or inactivation of the enzyme by a competing substrate; lack of co-factor to facilitate the reaction or lack of enzyme substrate.

The invention also provides methods of addressing the issue of rate limiting pathway reactions. The deficit of enzymatic activity in the rate-limiting pathway reaction may be addressed by providing a recombinant Clostridia microorganism adapted to exhibit at least one of i) an increase in activity of the enzyme or a functionally equivalent variant thereof or ii) availability of the co-factor involved in the rate-limiting pathway reaction. This results in an overall increase in the flux through the pathway.

Accordingly, the invention provides a method of producing a recombinant carboxydotrophic Clostridia microorganism adapted to exhibit increased flux through a fermentation pathway relative to a parental microorganism, the method comprising:
a) determining a rate limiting pathway reaction in the fermentation pathway;
b) identifying one or more enzymes, co-factors or both, which are involved in catalysing the rate-limiting pathway reaction;
c) transforming a parental microorganism to yield a recombinant microorganism adapted to exhibit at least one of i) increased activity of the one or more enzymes of b) or a functionally equivalent variant of any one or more thereof, or ii) increased availability of the one or more co-factors of b), when compared to a parental microorganism;
   wherein the fermentation pathway is capable of producing one or more fermentation products from a substrate comprising CO.

In a particular embodiment of the invention, the recombinant microorganism is adapted to do at least one of:
i) over-express the one or more enzymes involved in catalysing the rate-limiting pathway reaction or a functionally equivalent variant of any one or more thereof;
ii) express one or more exogenous enzymes involved in catalysing the rate-limiting pathway reaction or a functionally equivalent variant of any one or more thereof; or
iii) have an increased availability of the one or more co-factors involved in catalysing the rate-limiting pathway reaction.

In this way, the inventors have demonstrated a method to overcome at least one of i) a low or a lack of enzymatic activity or ii) a low or lack of availability of a co-factor in a way that increases the flux through a fermentation pathway and ultimately increases the efficiency of the fermentation.

In a particular embodiment of the invention, the one or more enzymes is selected from the group consisting of alcohol dehydrogenase (EC 1.1.1.1), aldehyde dehydrogenase (acylating) (EC 1.2.1.10), formate dehydrogenase (EC 1.2.1.2), formyl-THF synthetase (EC 6.3.2.17), methylene-THF dehydrogenase/formyl-THF cyclohydrolase (EC:6.3.4.3), methylene-THF reductase (EC 1.1, 1.58), CO dehydrogenase/acetyl-CoA synthase (EC 2.3.1.169), aldehyde ferredoxin oxidoreductase (EC 1.2.7.5), phosphotransacetylase (EC 2.3.1.8), acetate kinase (EC 2.7.2.1), CO dehydrogenase (EC 1.2.99.2), and hydrogenase (EC 1.12.7.2). Preferably, the microorganism of this embodiment is adapted to exhibit an increase in the flux through a fermentation pathway resulting in the production of ethanol.

In a particular embodiment of the invention, the one or more enzymes enzymes is selected from the group consisting of pyruvate:ferredoxin oxidoreductase (Pyruvate synthase) (EC 1.2.7.1), pyruvate:formate lyase (EC 2.3.1.54), acetolactate synthase (EC 2.2.1.6), acetolactate decarboxylase (EC 4.1.1.5), 2,3-butanediol dehydrogenase (EC 1.1.1.4), primary:seconday alcohol dehydrogenase (EC 1.1.1.1), formate dehydrogenase (EC 1.2.1.2), formyl-THF synthetase (EC 6.3.2.17), methylene-THF dehydrogenase/formyl-THF cyclohydrolase (EC:6.3.4.3), methylene-THF reductase (EC 1.1, 1.58), CO dehydrogenase/acetyl-CoA synthase (EC 2.3.1.169), CO dehydrogenase (EC 1.2.99.2), and hydrogenase (EC 1.12.7.2). Preferably, the microorganism of this embodiment is adapted to exhibit an increase in the flux through a fermentation pathway resulting in the production of 2,3-butanediol.

In a particular embodiment of the invention, the recombinant microorganism is adapted to express an exogenous nucleic acid, or over-express an endogenous nucleic acid, wherein said nucleic acid encodes an enzyme or a functionally equivalent variant thereof, or is involved in the biosynthesis of a co-factor, wherein said enzyme or co-factor is involved in catalysing the rate limiting pathway reaction.

Methods to produce recombinant microorganisms of use in the invention are described hereinafter.

The nucleic acids encoding enzymes described above would be known to one of skill in the art and could be easily identified using gene information databases such as NCBI, KEGG, UniProt.

The inventors have also surprisingly found that increasing the co-factor availability has an effect on the overall flux through the fermentation pathway. In these cases, a fermentation pathway or reaction within this fermentation pathway is dependent on and may be limited by the availability of a certain co-factor. The pool of a co-factor available for use in a reaction can be increased by altering the expression of proteins and genes involved in the biosynthesis pathway of this co-factor. As a result of increased co-factor availability, the reaction dependent on this co-factor is not limited anymore.

In a particular embodiment, the co-factor comprises tetrahydrofolate. As noted above, enzymes involved in the biosynthesis of such co-factor may be overexpressed, preferably by expressing or over-expressing the corresponding gene encoding the enzyme. Enzymes that are involved in the biosynthesis of tetrahydrofolate are detailed below. Accordingly, in a particular embodiment, the recombinant microorganism exhibits increased expression of GTP cyclohydrolase I (EC 3.5.4.16), alkaline phosphatase (EC 3.1.3.1), dihydroneopterin aldolase (EC 4.1.2.25), 2-amino-4-hydroxy-6-hydroxymethyldihydropteridine diphosphokinase (EC 2.7.6.3), dihydropteroate synthase (2.5.1.15), dihydropteroate synthase (EC 2.5.1.15), dihydrofolate synthase (EC 6.3.2.12), folylpolyglutamate synthase (6.3.2.17), dihydrofolate reductase (EC 1.5.1.3), thymidylate synthase (EC 2.1.1.45), dihydromonapterin reductase (EC 1.5.1.-). All involved in thf

In a particular embodiment, the co-factor comprises cobalamine (B₁₂). Enzymes that are involved in the biosynthesis of cobalamine are detailed below. Accordingly, in a particular embodiment, the recombinant microorganism exhibits increased expression of 5-aminolevulinate synthase (EC 2.3.1.37), 5-aminolevulinate:pyruvate aminotransferase (EC 2.6.1.43), adenosylcobinamide kinase/adenosylcobinamide-phosphate guanylyltransferase (EC 2.7.1.156/2.7.7.62), adenosylcobinamide-GDP ribazoletransferase (EC 2.7.8.26), adenosylcobinamide-phosphate synthase (EC 6.3.1.10), adenosylcobyric acid synthase (EC 6.3.5.10), alpha-ribazole phosphatase (EC 3.1.3.73), cob(I)alamin adenosyltransferase (EC 2.5.1.17), cob(II)yrinic acid a,c-diamide reductase (EC 1.16.8.1), cobalt-precorrin 5A hydrolase (EC 3.7.1.12), cobalt-precorrin-5B (C1)-methyltransferase (EC 2.1.1.195), cobalt-precorrin-7 (C15)-methyltransferase (EC 2.1.1.196), cobaltochelatase CobN (EC 6.6.1.2), cobyrinic acid a,c-diamide synthase (EC 6.3.5.9/6.3.5.11), ferritin (EC 1.16.3.1), glutamate-1-semialdehyde 2,1-aminomutase (EC 5.4.3.8), glutamyl-tRNA reductase (EC 1.2.1.70), glutamyl-tRNA synthetase (EC 6.1.1.17), hydroxymethylbilane synthase (EC 2.5.1.61), nicotinate-nucleotide-dimethylbenzimidazole phosphoribosyltransferase (EC 2.4.2.21), oxygen-independent coproporphyrinogen III oxidase (EC 1.3.99.22), porphobilinogen synthase (EC 4.2.1.24), precorrin-2 dehydrogenase/sirohydrochlorin ferrochelatase (EC 1.3.1.76/4.99.1.4), precorrin-2/cobalt-factor-2 C20-methyltransferase (EC 2.1.1.130/2.1.1.151), precorrin-3B synthase (EC 1.14.13.83), precorrin-3B C17-methyltransferase (EC 2.1.1.131), precorrin-4 C11-methyltransferase (EC 2.1.1.133), precorrin-6X reductase (EC 1.3.1.54), precorrin-6Y C5,15-methyltransferase (EC 2.1.1.132), precorrin-8W decarboxylase (EC 1.-.-.-), precorrin-8X methylmutase (EC 5.4.1.2), sirohydrochlorin cobaltochelatase (EC 4.99.1.3), threonine-phosphate decarboxylase (EC 4.1.1.81), uroporphyrinogen decarboxylase (EC 4.1.1.37), uroporphyrinogen III methyltransferase/synthase (EC 2.1.1.107/4.2.1.75)

The biosynthesis genes encoding the above-mentioned proteins would be known to one of skill in the art or could be easily identified using gene information databases.

Without wishing to be bound by theory, it is believed that an increase in the availability of a co-factor is achieved through over-expression of enzymes or genes involved in the biosynthesis pathway of said co-factor. As a result, reactions dependent on this co-factor are no longer limiting.

### Modification of Enzymes to Achieve Higher Activity

In a particular embodiment of the invention, the recombinant microorganism has undergone enzyme engineering to increase enzymatic activity of an enzyme capable of catalysing a rate-limiting pathway reaction. Enzyme engineering may include any genetic modification known to those of skill in the art including but not limited to deletion, insertion and substitution of one or more nucleotides. Suitable methods to achieve increased enzymatic activity will be known to one of skill in the art but by way of example, the method of enzyme engineering may be selected from the group consisting of directed evolution, knowledge based design, random mutagenesis methods, gene shuffling, codon optimization, use of site-specific libraries and use of site evaluation libraries.

### Recombinant Microorganism

The invention provides in a further aspect a recombinant carboxydotrophic Clostridia microorganism produced by the method as described above wherein the recombinant microorganism is adapted to exhibit increased flux through a fermentation pathway relative to a parental microorganism.

In particular embodiments, the increase in expression of the enzyme and/or the increase in availability of the co-factor is effected by the expression and/or overexpression of a nucleic acid encoding said enzyme or involved in the biosynthesis of said co-factor.

In a further aspect, the invention provides the use of a microorganism of the invention to increase the flux through a reaction pathway.

In one particular embodiment of the invention, the parental microorganism is selected from the group of carboxydotrophic Clostridia comprising Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum.

In a one embodiment, the microorganism is selected from a cluster of carboxydotrophic Clostridia comprising the species C. autoethanogenum, C. ljungdahlii, and “C. ragsdalei” and related isolates. These include but are not limited to strains C. autoethanogenum JAI-1^{T} (DSM10061) (Abrini et al., 1994), C. autoethanogenum LBS1560 (DSM19630) (WO/2009/064200), C. autoethanogenum, C. ljungdahlii PETC^{T} (DSM13528=ATCC 55383) (Tanner et al., 1993), C. ljungdahlii ERI-2 (ATCC 55380) (U.S. Pat. No. 5,593,886), C. ljungdahlii C-01 (ATCC 55988) (U.S. Pat. No. 6,368,819), C. ljungdahlii O-52 (ATCC 55989) (U.S. Pat. No. 6,368,819), or “C. ragsdalei P11^{T}” (ATCC BAA-622) (WO 2008/028055), and related isolates such as “C. coskatii” (US patent 2011/0229947) or “Clostridium sp. MT351” (Tyurin & Kiriukhin, 2012) and mutant strains thereof such as C. ljungdahlii OTA-1 (Tirado-Acevedo 0. Production of Bioethanol from Synthesis Gas Using Clostridium ljungdahlii. PhD thesis, North Carolina State University, 2010).

These strains form a subcluster within the Clostridial rRNA cluster I (Collins et al., 1994), having at least 99% identity on 16S rRNA gene level, although being distinct species as determined by DNA-DNA reassociation and DNA fingerprinting experiments (WO 2008/028055, US patent 2011/0229947).

The strains of this cluster are defined by common characteristics, having both a similar genotype and phenotype, and they all share the same mode of energy conservation and fermentative metabolism. The strains of this cluster lack cytochromes and conserve energy via an Rnf complex.

All strains of this cluster have a genome size of around 4.2 MBp (Köpke et al., 2010) and a GC composition of around 32% mol (Tanner et al., 1993; Abrini et al., 1994; Köpke et al., 2010) (WO 2008/028055; US patent 2011/0229947), and conserved essential key gene operons encoding for enzymes of Wood-Ljungdahl pathway (Carbon monoxide dehydrogenase, Formyl-tetrahydrofolate synthetase, Methylene-tetrahydrofolate dehydrogenase, Formyl-tetrahydrofolate cyclohydrolase, Methylene-tetrahydrofolate reductase, and Carbon monoxide dehydrogenase/Acetyl-CoA synthase), hydrogenase, formate dehydrogenase, Rnf complex (rnfCDGEAB), pyruvate:ferredoxin oxidoreductase, aldehyde:ferredoxin oxidoreductase (Köpke et al., 2010, 2011). The organization and number of Wood-Ljungdahl pathway genes, responsible for gas uptake, has been found to be the same in all species, despite differences in nucleic and amino acid sequences (Köpke et al., 2011).

The strains all have a similar morphology and size (logarithmic growing cells are between 0.5−0.7×3-5 μm), are mesophilic (optimal growth temperature between 30-37° C.) and strictly anaerobe (Tanner et al., 1993; Abrini et al., 1994)(WO 2008/028055). Moreover, they all share the same major phylogenetic traits, such as same pH range (pH 4-7.5, with an optimal initial pH of 5.5-6), strong autotrophic growth on CO containing gases with similar growth rates, and a metabolic profile with ethanol and acetic acid as main fermentation end product, with small amounts of 2,3-butanediol and lactic acid formed under certain conditions (Tanner et al., 1993; Abrini et al., 1994; Köpke et al., 2011)(WO differentiate in substrate utilization of various sugars (e.g. rhamnose, arabinose), acids (e.g. gluconate, citrate), amino acids (e.g. arginine, histidine), or other substrates (e.g. betaine, butanol). Some of the species were found to be auxotroph to certain vitamins (e.g. thiamine, biotin) while others were not. Reduction of carboxylic acids into their corresponding alcohols has been shown in a range of these organisms (Perez et al., 2012).

The traits described are therefore not specific to one organism like C. autoethanogenum or C. ljungdahlii, but rather general traits for carboxydotrophic, ethanol-synthesizing Clostridia. Thus, the invention can be anticipated to work across these strains, although there may be differences in performance.

In certain embodiments, the parental microorganism is selected from the group comprising Clostridium autoethanogenum, Clostridium ljungdahlii, and Clostridium ragsdalei. In one embodiment, the group also comprises Clostridium coskatii. In one particular embodiment, the parental microorganism is Clostridium autoethanogenum.

In one embodiment, the invention provides a recombinant microorganism adapted to express an enzyme or to increase availability of a co-factor where the enzyme expression or co-factor availability is dependent on expression of a nucleic acid. The recombinant microorganism may also express a nucleic acid construct or vector adapted to result in an increase in expression of the enzyme and/or availability of a co-factor. In one particular embodiment, the nucleic acid construct or vector is an expression construct or vector, however other constructs and vectors, such as those used for cloning are encompassed by the invention. In one particular embodiment, the expression construct or vector is a plasmid.

It will be appreciated that an expression construct/vector of the present invention may contain any number of regulatory elements in addition to the promoter as well as additional genes suitable for expression of further proteins if desired. In one embodiment the expression construct/vector includes one promoter. In another embodiment, the expression construct/vector includes two or more promoters. In one particular embodiment, the expression construct/vector includes one promoter for each gene to be expressed. In one embodiment, the expression construct/vector includes one or more ribosomal binding sites, preferably a ribosomal binding site for each gene to be expressed.

It will be appreciated by those of skill in the art that the nucleic acid sequences and construct/vector sequences described herein may contain standard linker nucleotides such as those required for ribosome binding sites and/or restriction sites. Such linker sequences should not be interpreted as being required and do not provide a limitation on the sequences defined.

Nucleic acids and nucleic acid constructs, including expression constructs/vectors of the invention may be constructed using any number of techniques standard in the art. For example, chemical synthesis or recombinant techniques may be used. Such techniques are described, for example, in Sambrook et al (Molecular Cloning: A laboratory manual, Cold Spring Harbor Laboratory Press, Cold Spring Harbor, N.Y., 1989). Essentially, the individual genes and regulatory elements will be operably linked to one another such that the genes can be expressed to form the desired proteins. Suitable vectors for use in the invention will be appreciated by those of ordinary skill in the art. However, by way of example, the following vectors may be suitable: pMTL80000 vectors, pIMP1, pJIR750, and the plasmids exemplified in the Examples section herein after.

It should be appreciated that nucleic acids as described herein may be in any appropriate form, including RNA, DNA, or cDNA.

### Method of Producing Recombinant Microorganisms

Methods of genetic modification of a parental microorganism include molecular methods such as heterologous gene expression, genome insertion or deletion, altered gene expression or inactivation of genes, or enzyme engineering methods as described herein. Such techniques are described, for example, in Sambrook et al (Molecular Cloning: A laboratory manual, Cold Spring Harbor Laboratory Press, Cold Spring Harbor, N.Y. 2001), Pleiss, (2011), Park, S, and Crochan, J. R., (2010, Protein engineering and design, CRC Press, ISBN 1420076582).

One or more exogenous nucleic acids may be delivered to a parental microorganism as naked nucleic acids or may be formulated with one or more agents to facilitate the transformation process (for example, liposome-conjugated nucleic acid, an organism in which the nucleic acid is contained). The one or more nucleic acids may be DNA, RNA, or combinations thereof, as is appropriate. Restriction inhibitors may be used in certain embodiments; see, for example Murray, N. E. et al. (2000) Microbial. Molec. Biol. Rev. 64, 412.)

The microorganisms of the invention may be prepared from a parental microorganism and one or more exogenous nucleic acids using any number of techniques known in the art for producing recombinant microorganisms. By way of example only, transformation (including transduction or transfection) may be achieved by electroporation, ultrasonication, polyethylene glycol-mediated transformation, chemical or natural competence, protoplast transformation, prophage induction or conjugation. Suitable transformation techniques are described for example in, Sambrook J, Fritsch E F, Maniatis T: Molecular Cloning: A laboratory Manual, Cold Spring Harbour Laboratory Press, Cold Spring Harbour, 1989.

Electroporation has been described for several carboxydotrophic acetogens as C. ljungdahlii (Köpke et al. 2010, Poc. Nat. Acad. Sci. U.S.A. 107: 13087-92; (Leang et al., 2011) PCT/NZ2011/000203; WO2012/053905), C. autoethanogenum (PCT/NZ2011/000203; WO2012/053905), Acetobacterium woodii (Straetz et al., 1994, Appl. Environ. Microbiol. 60:1033-37) or Moorella thermoacetica (Kita et al., 2012) and is a standard method used in many Clostridia such as C. acetobutylicum (Mermelstein et al., 1992, Biotechnology, 10, 190-195), C. cellulolyticum (Jennert et al., 2000, Microbiology, 146: 3071-3080) or C. thermocellum (Tyurin et al., 2004, Appl. Environ. Microbiol. 70: 883-890). Prophage induction has been demonstrated for carboxydotrophic acetogen as well in case of C. scatologenes (Prasanna Tamarapu Parthasarathy, 2010, Development of a Genetic Modification System in Clostridium scatologenes ATCC 25775 for Generation of Mutants, Masters Project Western Kentucky University), while conjugation has been described as method of choice for many Clostridia including Clostridium difficile (Herbert et al., 2003, FEMS Microbiol. Lett. 229: 103-110) or C. acetobuylicum (Williams et al., 1990, J. Gen. Microbiol. 136: 819-826) and could be used in a similar fashion for carboxydotrophic acetogens.

In certain embodiments, due to the restriction systems which are active in the microorganism to be transformed, it is necessary to methylate the nucleic acid to be introduced into the microorganism. This can be done using a variety of techniques, including those described below.

By way of example, in one embodiment, a recombinant microorganism of the invention is produced by a method comprising the following steps:
introduction into a shuttle microorganism of (i) of an expression construct/vector comprising a nucleic acid as described herein and (ii) a methylation construct/vector comprising a methyltransferase gene;
expression of the methyltransferase gene; isolation of one or more constructs/vectors from the shuttle microorganism; and, introduction of the one or more construct/vector into a destination microorganism.

In one embodiment, the methyltransferase gene of step B is expressed constitutively. In another embodiment, expression of the methyltransferase gene of step B is induced.

The shuttle microorganism is a microorganism, preferably a restriction negative microorganism, that facilitates the methylation of the nucleic acid sequences that make up the expression construct/vector. In a particular embodiment, the shuttle microorganism is a restriction negative E. coli, Bacillus subtillis, or Lactococcus lactis.

The methylation construct/vector comprises a nucleic acid sequence encoding a methyltransferase.

Once the expression construct/vector and the methylation construct/vector are introduced into the shuttle microorganism, the methyltransferase gene present on the methylation construct/vector is induced. Induction may be by any suitable promoter system although in one particular embodiment of the invention, the methylation construct/vector comprises an inducible lac promoter and is induced by addition of lactose or an analogue thereof, more preferably isopropyl-β-D-thio-galactoside (IPTG). Other suitable promoters include the ara, tet, or T7 system. In a further embodiment of the invention, the methylation construct/vector promoter is a constitutive promoter.

In a particular embodiment, the methylation construct/vector has an origin of replication specific to the identity of the shuttle microorganism so that any genes present on the methylation construct/vector are expressed in the shuttle microorganism. Preferably, the expression construct/vector has an origin of replication specific to the identity of the destination microorganism so that any genes present on the expression construct/vector are expressed in the destination microorganism.

Expression of the methyltransferase enzyme results in methylation of the genes present on the expression construct/vector. The expression construct/vector may then be isolated from the shuttle microorganism according to any one of a number of known methods. By way of example only, the methodology described in the Examples section described hereinafter may be used to isolate the expression construct/vector.

In one particular embodiment, both construct/vector are concurrently isolated.

The expression construct/vector may be introduced into the destination microorganism using any number of known methods. However, by way of example, the methodology described in the Examples section hereinafter may be used. Since the expression construct/vector is methylated, the nucleic acid sequences present on the expression construct/vector are able to be incorporated into the destination microorganism and successfully expressed.

It is envisaged that a methyltransferase gene may be introduced into a shuttle microorganism and over-expressed. Thus, in one embodiment, the resulting methyltransferase enzyme may be collected using known methods and used in vitro to methylate an expression plasmid. The expression construct/vector may then be introduced into the destination microorganism for expression. In another embodiment, the methyltransferase gene is introduced into the genome of the shuttle microorganism followed by introduction of the expression construct/vector into the shuttle microorganism, isolation of one or more constructs/vectors from the shuttle microorganism and then introduction of the expression construct/vector into the destination microorganism.

It is envisaged that the expression construct/vector and the methylation construct/vector as defined above may be combined to provide a composition of matter. Such a composition has particular utility in circumventing restriction barrier mechanisms to produce the recombinant microorganisms of the invention.

In one particular embodiment, the expression construct/vector and/or the methylation construct/vector are plasmids.

Persons of ordinary skill in the art will appreciate a number of suitable methyltransferases of use in producing the microorganisms of the invention. However, by way of example the Bacillus subtilis phage ΦT1 methyltransferase and the methyltransferase described in the Examples herein after may be used. In one embodiment, the methyltransferase has been described in WO/2012/053905.

Any number of constructs/vectors adapted to allow expression of a methyltransferase gene may be used to generate the methylation construct/vector. However, by way of example, the plasmid described in the Examples section hereinafter may be used.

### Methods of Production

As mentioned herein before, the invention also provides methods for the production of one or more products by fermentation of a substrate comprising CO.

In a particular embodiment, the substrate comprising CO is a gaseous substrate comprising CO. In a particular embodiment of any of the previous aspects, the substrate will typically contain a major proportion of CO, such as at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

The gaseous substrate may be a CO-containing waste gas obtained as a by-product of an industrial process, or from some other source such as from automobile exhaust fumes. In certain embodiments, the industrial process is selected from the group consisting of ferrous metal products manufacturing, such as a steel mill, non-ferrous products manufacturing, petroleum refining processes, gasification of coal, electric power production, carbon black production, ammonia production, methanol production and coke manufacturing. In these embodiments, the CO-containing gas may be captured from the industrial process before it is emitted into the atmosphere, using any convenient method. The CO may be a component of syngas (gas comprising carbon monoxide and hydrogen). The CO produced from industrial processes is normally flared off to produce CO₂ and therefore the invention has particular utility in reducing CO₂ greenhouse gas emissions and producing a biofuel. Depending on the composition of the gaseous CO-containing substrate, it may also be desirable to treat it to remove any undesired impurities, such as dust particles before introducing it to the fermentation. For example, the gaseous substrate may be filtered or scrubbed using known methods.

It will be appreciated that for growth of the bacteria and the production of products to occur, in addition to the CO-containing substrate gas, a suitable liquid nutrient medium will need to be fed to the bioreactor.

In particular embodiments of the method aspects, the fermentation occurs in an aqueous culture medium. In particular embodiments of the method aspects, the fermentation of the substrate takes place in a bioreactor.

The substrate and media may be fed to the bioreactor in a continuous, batch or batch fed fashion. A nutrient medium will contain vitamins and minerals sufficient to permit growth of the micro-organism used. Anaerobic media suitable for fermentation using CO are known in the art. For example, suitable media are described Biebel (2001). In one embodiment of the invention the media is as described in the Examples section herein after.

The fermentation should desirably be carried out under appropriate fermentation conditions for the production of the fermentation product to occur. Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition.

In addition, it is often desirable to increase the CO concentration of a substrate stream (or CO partial pressure in a gaseous substrate) and thus increase the efficiency of fermentation reactions where CO is a substrate. Operating at increased pressures allows a significant increase in the rate of CO transfer from the gas phase to the liquid phase where it can be taken up by the micro-organism as a carbon source for the production of fermentation. This in turn means that the retention time (defined as the liquid volume in the bioreactor divided by the input gas flow rate) can be reduced when bioreactors are maintained at elevated pressure rather than atmospheric pressure. The optimum reaction conditions will depend partly on the particular micro-organism of the invention used. However, in general, it is preferred that the fermentation be performed at pressure higher than ambient pressure. Also, since a given CO conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

By way of example, the benefits of conducting a gas-to-ethanol fermentation at elevated pressures has been described. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that one or more product is consumed by the culture.

The composition of gas streams used to feed a fermentation reaction can have a significant impact on the efficiency and/or costs of that reaction. For example, O2 may reduce the efficiency of an anaerobic fermentation process. Processing of unwanted or unnecessary gases in stages of a fermentation process before or after fermentation can increase the burden on such stages (e.g. where the gas stream is compressed before entering a bioreactor, unnecessary energy may be used to compress gases that are not needed in the fermentation). Accordingly, it may be desirable to treat substrate streams, particularly substrate streams derived from industrial sources, to remove unwanted components and increase the concentration of desirable components.

In certain embodiments a culture of a bacterium of the invention is maintained in an aqueous culture medium. Preferably the aqueous culture medium is a minimal anaerobic microbial growth medium. Suitable media are known in the art and described for example in U.S. Pat. Nos. 5,173,429 and 5,593,886 and WO 02/08438, and as described in the Examples section herein after.

Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

### EXAMPLES

### Example 1

### Identification of Bottlenecks

Fermentation pathways of carboxydotrophic bacteria such as C. autoethanogenum, C. ljungdahlii, or C. ragsdalei for production of ethanol and 2,3-butanediol were analyzed for bottlenecks using enzyme assays. For this, oxidoreductase reactions are particularly suitable, as they are coupled with one or more co-factors whose reduction or oxidation can be measured. A synthetic redox dye such as methylviologen or benzylviologen can be used for this purpose as well.

Oxidoreductase enzyme steps of the Wood-Ljungdahl pathway and fermentation pathways to ethanol and 2,3-butanediol were assayed to determine their activity. The enzymes in these pathways are involved in autotrophic growth including uptake and utilization of CO, CO₂, and H₂ gases as well as product formation.

The enzymes assayed and their activities are detailed in FIG. 1. All assays performed were tested using a synthetic redox dye as control, either methyl viologen (MV) or benzyl viologen (BV). Co-factors ferredoxin (Fd), NADH and NADPH or a combination thereof was tested as described below. Enzyme assays were performed using crude extracts from a fermentation as described below growing autotrophically on CO and hydrogen:

### Fermentation

Fermentations with C. autoethanogenum were carried out in 1.5 L bioreactors at 37° C. and CO-containing steel mill gas as sole energy and carbon source as described below. Fermentation media containing per litre: MgCl, CaCl₂ (0.5 mM), KCl (2 mM), H₃PO₄ (5 mM), Fe (100 μM), Ni, Zn (5 μM), Mn, B, W, Mo, Se (2 μM) was used for culture growth. The media was transferred into the bioreactor and autoclaved at 121° C. for 45 minutes. After autoclaving, the media was supplemented with Thiamine, Pantothenate (0.05 mg), Biotin (0.02 mg) and reduced with 3 mM Cysteine-HCl. To achieve anaerobicity the reactor vessel was sparged with nitrogen through a 0.2 μm filter. Prior to inoculation, the gas was switched to CO-containing steel mill gas, feeding continuously to the reactor. The feed gas composition was 2% H₂, 42% CO, 20% CO₂ and 36% N₂. The pH of the culture was maintained between 5 and 5.2.

### Harvesting of Cells

At the time of harvesting the cells (biomass of 3.9 g cells/1 fermentation broth), the gas consumption was 5 moles CO L⁻¹ day⁻¹ and 10 milimoles H₂ L⁻¹ day⁻¹, with the following metabolites produced: 14 g L⁻¹ day⁻¹ Acetate and 19.5 g L⁻¹ day⁻¹ Ethanol. The pH of the culture was adjusted to pH 6 with K₂CO₃ and the reactor chilled in an ice-water bath. Approximately 1.2 L of culture was collected on ice. The culture was divided between two 1-L centrifuge bottles (this and all subsequent steps were carried out in an anaerobic chamber to ensure anoxic conditions to avoid inactivation of the enzymes) and cells pelleted at 5000 rpm for 10 min. The supernatant was decanted, and residual liquid removed. Each pellet was resuspended in approximately 30 mL of 50 mM K PO₄ pH 7.0 with 10 mM DTT. Resuspensions were transferred to pre weighed 50-mL-Falcon-tubes and cells repelleted at maximum speed (5000 g) for 15 min. The tubes were removed from the anaerobic chamber and immediately frozen on liquid N₂ before assaying.

### Preparation of Crude Cell Extracts and Enzyme Assays

Cells were harvested from a continuous reactor under anoxic conditions. They were disrupted by three passes through a French press as described by Huang et al. (2012). Unbroken cells and cell debris were removed by centrifugation at 20,000×g and 4° C. for 30 min. The supernatant was used for enzyme assays. Except where indicated, all assays were performed at 37° C. in 1.5-ml anaerobic cuvettes closed with a rubber stopper filled with 0.8 ml reaction mixture and 0.7 ml N₂ or H₂ or CO at 1.2×10⁵ Pa as described by Huang et al. (2012). Enzymes were assayed as described below or by (Huang et al., (2012). After the start of the reaction with enzyme, the reduction of NAD(P)⁺ or NAD⁺ was monitored spectrophotometrically at 340 nm (ε=6.2 mM⁻¹ cm⁻¹) or at 380 nm (ε=1.2 mM⁻¹ cm⁻¹), C. pasteurianum ferredoxin reduction at 430 nm (ε_{Δox-red}≈13.1 mM⁻¹ cm⁻¹), methyl viologen reduction at 578 nm (ε=9.8 mM⁻¹ cm⁻¹) and benzyl viologen reduction at 578 nm (ε=8.6 mM⁻¹ cm⁻¹).

CO dehydrogenase was measured using an assay mixture that contained 100 mM Tris/HCl (pH 7.5), 2 mM DTT and about 30 μM ferredoxin and/or 1 mM NAD⁺ or 1 mM NADP⁺. The gas phase was 100% CO.

Hydrogenase activity was measured using an assay mixture of 100 mM Tris/HCl (pH 7.5) or 100 mM potassium phosphate, 2 mM DTT and, 25 μM ferredoxin and/or 1 mM NADP⁺ and/or 10 mM methyl viologen. The gas phase was 100% H₂.

Formate-Hydrogen lyase activity for reduction of CO₂ with H₂ to formate was measured with an assay mixture containing 100 mM potassium phosphate, 2 mM DTT, and 30 mM [¹⁴C]K₂CO₃ (24,000 dpm/mmol). The gas phase was 100% H₂. The serum bottles were continuously shaken at 200 rpm to ensure equilibration of the gas phase with the liquid phase. After start of the reaction with enzyme, 100 μl liquid samples were withdrawn every 1.5 min and added into a 1.5-ml safe seal micro tube containing 100 μl of 150 mM acetic acid to stop the reaction by acidification. The 200 μl mixture was then incubated at 40° C. for 10 min with shaking at 1,400 rpm in a Thermomixer to remove all ¹⁴CO₂ leaving behind the ¹⁴C-formate formed. Subsequently, 100 μl of the mixture was added to 5 ml of Quicksave A scintillation fluid (Zinsser Analytic, Frankfurt, Germany) and analyzed for ¹⁴C radioactivity in a Beckman LS6500 liquid scintillation counter (Fullerton, Calif.).

Formate dehydrogenase measurement was carried out with an assay mixtures containing 100 mM Tris/HCl (pH 7.5) or 100 mM potassium phosphate, 2 mM DTT, 20 mM formate and, where indicated 25 μM ferredoxin, 1 mM NADP⁺, 1 mM NAD⁺ and/or 10 mM methyl viologen. The gas phase was 100% N₂.

Methylene-H₄F dehydrogenase was measured using an assay mixture containing 100 mM MOPS/KOH (pH 6.5), 50 mM 2-mercaptoethanol, 0.4 mM tetrahydrofolate, 10 mM formaldehyde and 0.5 mM NADP⁺ or 0.5 mM NAD⁺. The gas phase was 100% N₂.

Methylene-H4F reductase was assayed under the following conditions. The assay mixtures contained 100 mM Tris/HCl (pH 7.5), 20 mM ascorbate, 10 μM FAD. 20 mM benzyl viologen and 1 mM methyl-H4F. Before start of the reaction with enzyme, benzyl viologen was reduced to an ΔA555 of 0.3 with sodium dithionite.

Aldehyde:ferredoxin oxidoreductase was assayed using a mixture containing 100 mM Tris/HCl (pH 7.5), 2 mM DTT, 1.1 mM acetaldehyde, and about 25 μM ferredoxin. The gas phase was 100% N2.

CoA acetylating acetaldehyde dehydrogenase was measured using a mixture contained 100 mM Tris/HCl (pH 7.5), 2 mM DTT, 1.1 mM acetaldehyde, 1 mM coenzyme A, and 1 mM NADP+ or 1 mM NAD+. The gas phase was 100% N2.

Alcohol and butanediol dehydrogenases were measured in an assay with 100 mM potassium phosphate (pH 6), 2 mM DTT, 1.1 mM acetaldehyde or acetoin respectively and 1 mM NADPH or 1 mM NADH. The gas phase was 100% N2.

Ferredoxin was purified from C. pasteurianum as described by Schönheit, Wäscher, & Thauer (1978).

### Results

All oxidoreductase reactions in the pathways to ethanol and 2,3-butanediol of carboxydotrophic bacterium C. autoethanogenum were assayed and successfully detected, with the exception of the methylene-THF reductase which the inventors believe requires an as yet unknown coupling site (Köpke et al., 2010; Poehlein et al., 2012), and activity of this enzyme couldn't be detected in other organisms previously. Results are provided in FIGS. 1 and 2. This data was used to analyze and determine bottlenecks in these pathways that would typically occur during a fermentation process.

### Example 2

### Bottleneck for Ethanol Production

As seen in the ethanol fermentation pathway depicted in FIG. 1, the bottleneck for ethanol production is the alcohol dehydrogenase reaction. While all other measured reactions showed at least an activity of 1.1 U/mg, the alcohol dehydrogenase reaction step has only a total activity of 0.35 U/mg (or 31%), 0.2 U/mg (18%) with NADH and 0.15 U/mg (13%) with NADPH. This is 69% less than all other reactions in the pathway. In a similar fashion, the aldehyde dehydrogenase reaction had only a total activity of 0.16 U/mg (14%), 0.08 U/mg (7%) with NADH and 0.08 U/mg (7%) with NADPH. This is 86% less than all other reactions in the pathway. This reaction however can be bypassed via acetate and the aldehyde:ferredoxin oxidoreductase (AOR) which has an activity of 1.9 U/mg and has the advantage of yielding ATP through substrate level phosphorylation in the acetate kinase reaction thus providing more energy to the cell. To go at least some way towards overcoming this bottleneck and increasing efficiency of a fermentation reaction, an endogenous alcohol and/or aldehyde dehydrogenase enzyme may be overexpressed in a recombinant microorganism, or an exogenous alcohol and/or aldehyde dehydrogenase enzyme may be introduced and expressed.

### Example 3

### Increasing the Flux Through an Ethanol Production Pathway by Removing Bottlenecks

The reactions catalysing the conversion of acetyl-coA to acetaldehyde and from acetaldehyde to ethanol have been identified to be the rate limiting steps in ethanol formation in C. autoethanogenum, C. ljungdahlii, or C. ragsdalei. This can be overcome by either

i. overexpressing the native bifunctional alcohol/aldehyde dehydrogenase,
ii. expressing a heterologous bifunctional alcohol/aldehyde dehydrogenase, or
iii. expressing a heterologous aldehyde dehydrogenase and an alcohol dehydrogenase.
These outcomes can be achieved by using the methods described below.
Overexpressing the Native Bifunctional Alcohol/Aldehyde Dehydrogenase in C. autoethanogenum

It was chosen to overexpress the native bifunctional alcohol/aldehyde dehydrogenase gene of C. autoethanogenum (Sequence ID: 1) and express a heterologous bifunctional alcohol/aldehyde dehydrogenase gene of C. acetobutylicum (Genbank nucleic acid ID: CP002661.1 and amino acid sequence ID: AEI34903.1) in C. autoethanogenum.

Genetic modifications were carried out using a plasmid pMTL83155 containing 487 bp promoter sequence of C. autoethanogenum phosphate acetyltransferase gene of C. autoethanogenum between NotI and NdeI restriction enzyme sites (as described in WO2012053905). This plasmid was methylated in vivo using a novel methyltransferase and then transformed into C. autoethanogenum.

### Design, Synthesis and Cloning of Codon Altered C. Autoethanogenum Bifunctional Alcohol/Aldehyde Dehydrogenase Gene

The nucleic acid sequence of C. autoethanogenum bifunctional alcohol/aldehyde dehydrogenase gene was codon altered to suit to other Clostridia (Sequence ID: 2) and synthesized. This is done to reduce the probability of homologous recombination between the chromosomal and episomal copies of the gene. The codon altered C. autoethanogenum bifunctional alcohol/aldehyde dehydrogenase gene shares 81% sequence identity with that of the unaltered one. The codon altered gene is isolated using NdeI and NheI restriction enzymes. The 2613 bp fragment is gel extracted using ZYMO Gel Extraction kit. The plasmid pMTL83155 is also treated with NdeI and NheI restriction enzymes followed by treatment with FASTAP alkaline phosphatase (Fermentas). The cut and phosphatase treated plasmid is cleaned using ZYMO Clean and Concentrate kit. Ligation is set with the cut insert and vector using T4 DNA ligase (Fermentas) for 1 h at 16° C. following which the ligation mix is used to transform E. coli TOP10 (Life Technologies). The TOP10 colonies are screened for plasmid with correct insert by plasmid isolation (ZYMO Plasmid Prep kit), restriction digestion with NdeI/NheI enzymes and finally by sequencing.

The correct plasmid, pMTL83155-cod.alt.naBiAADH, is introduced into E. coli XL1-Blue MRF′ Kan strain already containing plasmid pGS20m with methyltransferase gene.

Cloning C. acetobutylicum Bifunctional Alcohol/Aldehyde Dehydrogenase Gene

The genomic DNA from C. acetobutylicum is isolated using Purelink Genomic DNA mini kit from Life Technologies, according to the manufacturer's instruction. The C. acetobutylicum bifunctional alcohol/aldehyde dehydrogenase gene is PCR amplified using primers caBiAADH-F (Sequence ID: 7) and caBiAADH-R (Sequence ID: 8) and iProof DNA polymerase (BioRad). The primers contain NdeI and NheI restriction enzyme sites. The 2589 bp PCR product is cleaned using ZYMO Clean and Concentrate kit. The PCR product and plasmid pMTL83155 is treated with NdeI and NheI restriction enzymes (Fermentas). The plasmid is further treated with FASTAP alkaline phosphatase (Fermentas). The cut and phosphatase treated plasmid and cut PCR product are cleaned using ZYMO Clean and Concentrate kit. Ligation is set with the cut insert and vector using T4 DNA ligase (Fermentas) for 1 h at 16° C. following which the ligation mix is used to transform E. coli TOP10 (Life Technologies). The TOP10 colonies are screened for plasmid with correct insert by plasmid isolation (ZYMO Plasmid Prep kit), restriction digestion with NdeI/NheI enzymes and finally by sequencing.

The correct plasmid, pMTL83155-caBiAADH, is introduced into E. coli XL1-Blue MRF′ Kan strain already containing plasmid pGS20m with methyltransferase gene.

Transformation of C. autoethanogenum:

### Methylation of DNA:

A hybrid methyltransferase gene fused to an inducible lac promoter (SEQ ID No. 27 from WO2012053905) was designed, by alignment of methyltransferase genes from C. autoethanogenum, C. ljungdahlii, and C. ragsdalei, as described in U.S. patent application Ser. No. 13/049,263. Expression of the methyltransferase results in a protein having the sequence of SEQ ID No. 28 from WO2012053905. The hybrid methyltransferase gene was chemically synthesized and cloned into vector pGS20 (ATG:biosynthetics GmbH, Merzhausen, Germany—SEQ ID No. 29 from WO2012053905 using EcoRI. The resulting methylation plasmid pGS20-methyltransferase was introduced into E. coli XL1-Blue MRF′ Kan strain (Stratagene) and this transformant was transformed again with plasmids pMTL83155-cod.alt.naBiAADH and pMTL83155-caBiAADH. In vivo methylation was induced by addition of 1 mM IPTG, and methylated plasmids were isolated (Qiagen min prep kit) and used for electroporating C. autoethanogenum.

### Electroporation:

During the complete transformation experiment, C. autoethanogenum was grown in YTF media (Tab. 2) in the presence of reducing agents and with 30 psi steel mill waste gas (collected from New Zealand Steel site in Glenbrook, NZ; composition: 44% CO, 32% N₂, 22% CO₂, 2% H₂) at 37° C. using standard anaerobic techniques described by Hungate (1969) and Wolfe (1971).

TABLE 2
YTF media

| Media component | per L of Stock | |
|---|---|---|
| Yeast extract | 10 | g |
| Tryptone | 16 | g |
| Sodium chloride | 0.2 | g |
| Fructose | 10 | g |
| Distilled water | To 1 | L |
| Reducing agent stock | per 100 mL of stock | |
| NaOH | 0.9 | g |
| Cystein•HCl | 4 | g |
| Na2S | 4 | g |
| Distilled water | To 100 | mL |

To make competent cells, a 50 ml culture of C. autoethanogenum was subcultured to fresh YTF media for 5 consecutive days. These cells were used to inoculate 50 ml YTF media containing 40 mM DL-threonine at an OD_{600 nm} of 0.05. When the culture reached an OD_{600 nm} of 0.5, the cells were incubated on ice for 30 minutes and then transferred into an anaerobic chamber and harvested at 4,700×g and 4° C. The culture was twice washed with ice-cold electroporation buffer (270 mM sucrose, 1 mM MgCl2, 7 mM sodium phosphate, pH 7.4) and finally suspended in a volume of 600 μl fresh electroporation buffer. This mixture was transferred into a pre-cooled electroporation cuvette with a 0.4 cm electrode gap containing 2 μg of the methylated plasmid mix and 1 μl Type 1 restriction inhibitor (Epicentre Biotechnologies) and immediately pulsed using the Gene pulser Xcell electroporation system (Bio-Rad) with the following settings: 2.5 kV, 600Ω, and 25 μF. Time constants of 3.7-4.0 ms were achieved. The culture was transferred into 5 ml fresh YTF media. Regeneration of the cells was monitored at a wavelength of 600 nm using a Spectronic Helios Epsilon Spectrophotometer (Thermo) equipped with a tube holder. After an initial drop in biomass, the cells started growing again. Once the biomass doubled from that point, about 200 μl of culture was spread on YTF-agar plates and PETC agar plates containing 5 g/l fructose (Table 3) (both containing 1.2% Bacto™ Agar (BD) and 15 μg/ml Thiamphenicol). Colonies are seen after 3-4 days of incubation with 30 psi steel mill gas at 37° C.

TABLE 3
PETC media (ATCC media 1754; atcc.org/Attachments/2940.pdf)

| Media component | Concentration per 1.0 L of media | |
|---|---|---|
| NH₄Cl | 1 | g |
| KCl | 0.1 | g |
| MgSO₄•7H₂O | 0.2 | g |
| NaCl | 0.8 | g |
| KH₂PO₄ | 0.1 | g |
| CaCl₂ | 0.02 | g |
| Trace metal solution | 10 | ml |
| Wolfe's vitamin solution | 10 | ml |
| Yeast Extract | 1 | g |
| Resazurin (2 g/L stock) | 0.5 | ml |
| MES | 2 | g |
| Reducing agent | 0.006-0.008% | (v/v) |
| Distilled water | Up to 1 L, pH 5.5 (adjusted with HCl) | |
| Wolfe's vitamin solution | per L of Stock | |
| Biotin | 2 | mg |
| Folic acid | 2 | mg |
| Pyridoxine hydrochloride | 10 | mg |
| Thiamine•HCl | 5 | mg |
| Riboflavin | 5 | mg |
| Nicotinic acid | 5 | mg |
| Calcium D-(+)-pantothenate | 5 | mg |
| Vitamin B₁₂ | 0.1 | mg |
| p-Aminobenzoic acid | 5 | mg |
| Thioctic acid | 5 | mg |
| Distilled water | To 1 | L |
| Trace metal solution | per L of stock | |
| Nitrilotriacetic Acid | 2 | g |
| MnSO₄•H₂O | 1 | g |
| Fe (SO₄)₂(NH₄)₂•6H₂O | 0.8 | g |
| CoCl₂•6H₂O | 0.2 | g |
| ZnSO₄•7H₂O | 0.2 | mg |
| CuCl₂•2H₂O | 0.02 | g |
| NaMoO₄•2H₂O | 0.02 | g |
| Na₂SeO₃ | 0.02 | g |
| NiCl₂•6H₂O | 0.02 | g |
| Na₂WO₄•2H₂O | 0.02 | g |
| Distilled water | To 1 | L |
| Reducing agent stock | per 100 | mL of stock |
| NaOH | 0.9 | g |
| Cystein•HCl | 4 | g |
| Na2S | 4 | g |
| Distilled water | To 100 | ml |

The colonies are streaked on fresh PETC agar plates also containing 5 g/L fructose and 15 μg/ml Thiamphenicol. After 2 days of incubation with 30 psi steel mill gas at 37° C. single colonies single colonies are picked into 2 ml PETC liquid media containing 5 g/l fructose and 15 μg/ml Thiamphenicol. When growth occurs, the culture volume is sequentially scaled up to 5 ml, 25 ml and then to 50 ml PETC media containing 5 g/l fructose, 15 μg/ml Thiamphenicol and 30 psi steel mill gas as carbon source.

The identity and the presence of plasmid in transformants are confirmed by PCR with primers fD1 (Sequence ID: 3) and rP2 (Sequence ID: 4); naBi-f (Sequence ID: 5) and naBi-r (Sequence ID: 6) for plasmid pMTL83155-cod.alt.naBiAADH and primers caBiAADH-F (Sequence ID: 7) and caBiAADH-R (Sequence ID: 8) for pMTL83155-caBiAADH, respectively.

Fermentation Experiment with C. Autoethanogenum Containing pMTL83155-cod.alt.naBiAADH and pMTL83155-caBiAADH

Fermentations are carried out in 1.5 L bioreactors at 37° C. and CO-containing steel mill gas as sole energy and carbon source as described below. A defined medium containing per litre: MgCl, CaCl₂ (0.5 mM), KCl (2 mM), H₃PO₄ (5 mM), Fe (100 μM), Ni, Zn (5 μM), Mn, B, W, Mo, Se (2 μM) is used for culture growth. The media is transferred into the bioreactor and autoclaved at 121° C. for 45 minutes. After autoclaving, the medium is supplemented with Thiamine, Pantothenate (0.05 mg), Biotin (0.02 mg) and reduced with 3 mM Cysteine-HCl. To achieve anaerobicity the reactor vessel is sparged with nitrogen through a 0.2 μm filter. Prior to inoculation, the gas is switched to CO-containing steel mill gas, feeding continuously to the reactor. The feed gas composition is 2% H₂ 42% CO 20% CO₂ 36% N₂. The pH of the culture is maintained between 5 and 5.2. The gas flow is initially set at 80 ml/min, increasing to 200 ml/min during mid-exponential phase, while the agitation is increased from 200 rpm to 350. Na₂S is dosed into the bioreactor at 0.25 ml/hr. Once the OD600 reached 0.5, the bioreactor is switched to a continuous mode at a rate of 1.0 ml/min (Dilution rate 0.96 d⁻¹). When the growth is stable, the reactor is spiked with 10 g/L racemic mix of acetoin. Media samples are taken to measure the biomass and metabolites by HPLC.

Analysis of metabolites is performed by HPLC using an Agilent 1100 Series HPLC system equipped with a RID operated at 35° C. (Refractive Index Detector) and an Alltech IOA-2000 Organic acid column (150×6.5 mm, particle size 5 μm) kept at 32° C. Slightly acidified water is used (0.005 M H₂SO₄) as mobile phase with a flow rate of 0.25 ml/min. To remove proteins and other cell residues, 400 μl samples are mixed with 100 μl of a 2% (w/v) 5-Sulfosalicylic acid and centrifuged at 14,000×g for 3 min to separate precipitated residues. 10 μl of the supernatant are then injected into the HPLC for analyses of key metabolites like ethanol, acetate, 2,3-butanediol and lactate.

The rate-limiting reaction diverting acetyl-coA to ethanol in C. autoethanogenum is relieved due to the overexpression of codon altered native bifunctional alcohol/aldehyde dehydrogenase gene of C. autoethanogenum or because of expression of a heterologous C. acetobutylicum bifunctional alcohol/aldehyde dehydrogenase gene. Thus an enhanced production flux to ethanol would be expected and a higher ethanol titer is produced in these genetically modified C. autoethanogenum strains.

Expression of a Heterologous Aldehyde Dehydrogenase and an Alcohol Dehydrogenase in C. autoethanogenum

For this we chose to overexpress the heterologous NAD/NADH dependant aldehyde dehydrogenase gene from Zymomonas mobilis (GenBank nucleic acid sequence ID NC_{—}006526.2 and amino acid sequence ID YP_{—}163331.1) and a NAD/NADH dependant alcohol dehydrogenase from C. beijerinckii (GenBank nucleic acid sequence ID CP000721.1 and amino acid sequence ID ABR35947.1) in C. autoethanogenum.

Design, Synthesis and Cloning of Codon Optimized Zymomonas Mobilis Aldehyde dehydrogenase gene

The nucleic acid sequence of Zymomonas mobilis aldehyde dehydrogenase gene is codon optimized for maximum expression in Clostridia The codon altered C. autoethanogenum bifunctional alcohol/aldehyde dehydrogenase gene shares 81% sequence identity with that of the unaltered one. The codon optimized gene is isolated using NdeI (supplied by Fermentas) and NheI (supplied by Fermentas) restriction enzymes. The 1152 bp fragment is gel extracted using ZYMO Gel Extraction kit. The plasmid pMTL83155 is also treated with NdeI and NheI restriction enzymes followed by treatment with FASTAP alkaline phosphatase (Fermentas). The cut and phosphatase treated plasmid is cleaned using ZYMO Clean and Concentrate kit. Ligation is set with the cut insert and vector using T4 DNA ligase (Fermentas) following which the ligation mix is used to transform E. coli TOP10 (Life Technologies). The TOP10 colonies are screened for plasmid with correct insert by plasmid isolation (ZYMO Plasmid Prep kit), restriction digestion with NdeI/NheI enzymes and finally by sequencing.

The correct plasmid, pMTL83155-zmAld, is introduced into E. coli XL1-Blue MRF′ Kan strain already containing plasmid pGS20m with methyltransferase gene as explained above.

### Cloning C. Beijerinckii Alcohol Dehydrogenase Gene

The genomic DNA from C. beijerinckii is isolated using Purelink Genomic DNA mini kit from Life Technologies, according to the manufacturer's instruction. The C. beijerinckii alcohol dehydrogenase gene is PCR amplified using primers cbAdh-F (Sequence ID: 9) and cbAdh-R (Sequence ID: 10) and iProof DNA polymerase (BioRad). The primers contain NdeI and NheI restriction enzyme sites. The 2589 bp PCR product is cleaned using ZYMO Clean and Concentrate kit. The PCR product and plasmid pMTL83155 is treated with NdeI and NheI restriction enzymes (Fermentas). The plasmid is further treated with FASTAP alkaline phosphatase (Fermentas). The cut and phosphatase treated plasmid and cut PCR product are cleaned using ZYMO Clean and Concentrate kit. Ligation is set with the cut insert and vector using T4 DNA ligase (Fermentas) for 1 h at 16° C. following which the ligation mix is used to transform E. coli TOP10 (Life Technologies). The TOP10 colonies are screened for plasmid with correct insert by plasmid isolation (ZYMO Plasmid Prep kit), restriction digestion with NdeI/NheI enzymes and finally by sequencing.

The correct plasmid, pMTL83155-cbAdh, is introduced into E. coli XL1-Blue MRF′ Kan strain already containing plasmid pGS20m with methyltransferase gene.

Cloning Codon Optimized Zymomonas Mobilis Aldehyde Dehydrogenase and C. Beijerinckii Alcohol Dehydrogenase Gene into One Plasmid

The codon optimized Zymomonas mobilis aldehyde dehydrogenase and C. beijerinckii alcohol dehydrogenase genes are assembled between suitable restriction sites as explained earlier on pMTL85155 plasmid to form an operon under the phosphate acetyltransferase promoter of C. autoethanogenum. The resulting plasmid, pMTL83155-zmAld-cbAdh, is introduced into E. coli XL1-Blue MRF′ Kan strain already containing plasmid pGS20m with methyltransferase gene

Transformation of C. autoethanogenum

Plasmids pMTL83155-zmAld, pMTL83155-cbAdh and pMTL83155-zmAld-cbAdh are all introduced into C. autoethanogenum by electroporation and resulting colonies screened as explained above.

Fermentation Experiment with C. Autoethanogenum Transformants Containing pMTL83155-zmAld, pMTL83155-cbAdh and pMTL83155-zmAld-cbAdh

Fermentation is carried out as explained in Example 1. The metabolites at different stages of fermentation are analysed by HPLC for ethanol, acetate, 2,3-butanediol and lactate.

Similar to the expression of bifunctional alcohol/aldehyde dehydrogenase, the rate limiting steps diverting acetyl-coA to ethanol in C. autoethanogenum is relieved due to the expression of codon optimized Zymomonas mobilis aldehyde dehydrogenase gene and C. beijerinckii alcohol dehydrogenase either individually or together in an operon. Thus an enhanced production flux to ethanol would be expected and a higher ethanol titer is produced in these genetically modified C. autoethanogenum strains.

### Example 4

### Bottleneck for 2,3-Butanediol Production

As seen in FIG. 2, the bottleneck for 2,3-butanediol production is the reaction from acetyl CoA to pyruvate catalysed by the pyruvate:ferredoxin reductase (PFOR) enzyme. While all other measured reactions showed at least an activity of 1.1 U/mg, this rate limiting reaction exhibited an enzyme activity of only 0.11 U/mg (10%) in the presence of Ferredoxin. This is 90% less than all other reactions in the pathway. To go at least some way towards overcoming this bottleneck and increase the product yield from the fermentation, an endogenous PFOR enzyme may be overexpressed or an exogenous PFOR enzyme may be introduced and expressed.

### Example 5

### Increasing the Flux Through the 2,3-Butanediol Production Pathway by Removing Bottlenecks

The reaction catalysing the conversion of acetyl-coA to pyruvate has been identified in FIG. 2 to be the rate limiting steps in 2,3-butanediol formation in C. autoethanogenum, C. ljungdahlii, or C. ragsdalei. This can be overcome by overexpressing the gene that encodes the pyruvate: ferredoxin oxidoreductase (PFOR) in C. autoethanogenum. The gene is synthesised with codons to express a protein with the amino acid sequence of that found natively in C. autoethanogenum (SEQ ID NO: 11).

The gene is codon-optimized and synthesized in order to reduce homology to the native gene and avoid unwanted integration events and minimise issues with expression (SEQ ID NO: 12). The gene is flanked by restriction enzyme cut sites, XbaI (3′-end) and NheI (5′-end) for subcloning into pMTL83155. The synthesized construct and pMTL83155 are digested with XbaI and NheI (Fermentas), and the pyruvate: ferredoxin oxidoreductase gene is ligated into pMTL83155 with T4 DNA ligase (Fermentas). The ligation mix is used to transform E. coli TOP10 (Invitrogen, LifeTechnologies) and colonies containing the desired plasmid are identified by plasmid miniprep (Zymo Research) and restriction digestion (Fermentas). The desired plasmid is methylated and transformed in C. autoethanogenum as described in example 3. Successful transformants are identified by thiamphenicol resistance and PCR analysis with primers repHF (SEQ ID NO: 13) and CatR (SEQ ID NO: 14) which will yield a 1584 base pair product when the plasmid is present.

Transformants identified as containing the desired plasmid are grown in serum bottles containing PETC-MES media in the presence of mill gas, and their metabolite production, measured by HPLC analysis, is compared to that of a parent organism not harbouring the plasmid. The PFOR activity in the transformed strain is also measured in crude extracts (as described in Example 1) to confirm that the observed bottleneck in the parent strain is alleviated. Overexpression of PFOR increases the overall activity within the cell, alleviating the bottleneck in the pathway, and leading to an increase in the flux through pyruvate, and an increase in 2,3-butanediol production.

### Example 6

### Bottleneck to Increase Acetyl-CoA Precursor for Increase of Overall Product Yield

As seen in FIGS. 1 and 2, gases CO and H₂ are readily utilized by the carboxydotrophic bacteria via carbon monoxide dehydrogenase, hydrogenase, and/or formate:hydrogen lyase with an activity of 2.7 and 2.4 U/mg respectively. However, the measured enzymes of the methyl branch of the Wood-Ljungdahl pathway (formate dehydrogenase, methylene-THF dehydrogenase) show only around 1.1 U/mg activity. To increase the level of acetyl-CoA, the precursor for all downstream products, endogenous enzymes of the methyl branch of the Wood-Ljungdahl pathway (Formate dehydrogenase, Formyl-THF synthetase, Methylene-THF, dehydrogenase/Formyl-THF cyclohydrolase, Methylene-THF reductase, Acetyl-CoA synthase) may be overexpressed or exogenous enzymes with substantially the same function may be introduced and expressed. Another strategy would be to increase the availability of required co-factors tetrahydrofolate and cobalamine by increasing expression of their biosynthesis genes.

### Example 7

### Relieving Last Bottlenecks for Optimized System to Achieve Better Production and Growth Rates

Previous examples describe how to optimize flux through the system including the Wood-Ljungdahl pathway and the ethanol and 2,3-Butanediol fermentation pathways to match the activity of the carbon monoxide dehydrogenase, hydrogenase, and/or formate:hydrogen lyase with an activity of 2.7 and 2.4 U/mg respectively.

Having addressed these previous bottlenecks, the inventors now turn to a further rate-limiting pathway reaction; the aldehyde:ferredoxin oxidoreducatse (AOR) reaction (see FIG. 1). This reaction has an activity of 1.9 U/mg, which is about 30% lower than the activity of the carbon monoxide dehydrogenase. As both the aldehyde:ferredoxin oxidoreductase (AOR) and the carbon monoxide dehydrogenase are one of the few enzymes that use ferredoxin as co-factor, it is particularly important to match the activities of both. This is because there is a finite pool of ferredoxin so the oxidation and reduction reactions cycling it should be balanced so as to ensure maximum efficiency of the cycle. The inventors have found that this can be achieved by overexpressing the aldehyde:ferredoxin oxidoreductase gene (AOR1—SEQ ID No. 16). Overexpression improved ethanol production by 31%. Relieving this bottleneck not only improved ethanol production by fermentation, but also improved growth rate of the organism as the ferredoxin pool is better balanced. Results are described in detail below.

### Construction of AOR1 Expression Plasmid

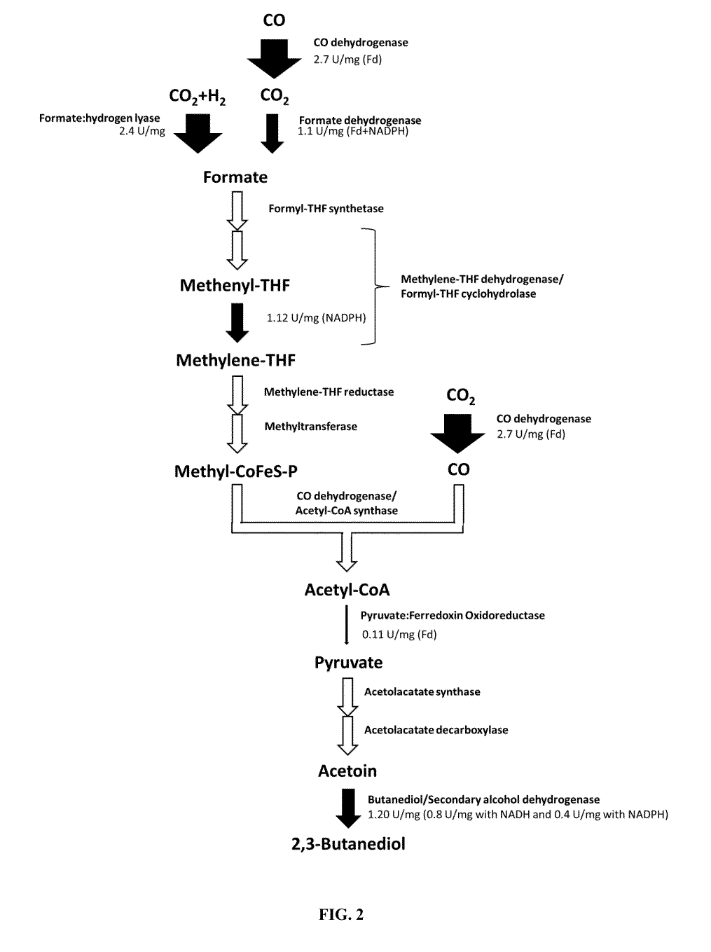

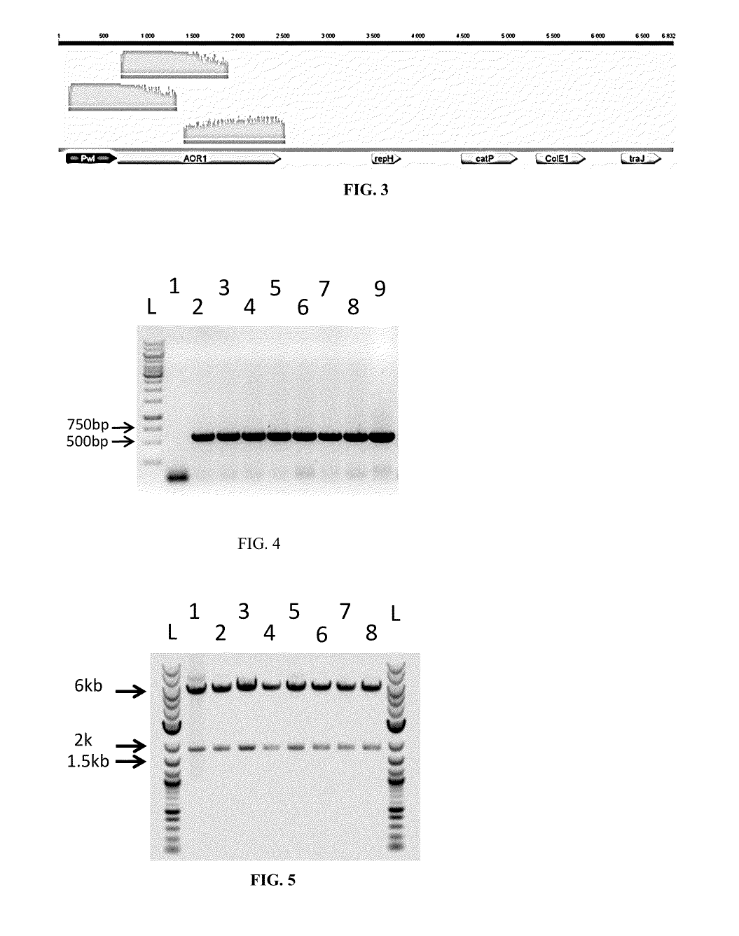

The DNA sequences of Wood-Ljungdahl promoter (P_{WL}) (Seq. ID. 15) and aldehyde::ferredoxin oxidoreductase 1 gene (AOR1) (Seq, ID. 16) were amplified by PCR with oligonucleotides in Table 4 using Phusion High Fidelity DNA Polymerase (New England Biolabs) from genomic DNA of Clostridum autoethanogenum DSM10061. Genomic DNA was isolated using a modified method by Bertram and Dune (1989). A 100-ml overnight culture was harvested (6,000×g, 15 min, 4° C.), washed with potassium phosphate buffer (10 mM, pH 7.5) and suspended in 1.9 ml STE buffer (50 mM Tris-HCl, 1 mM EDTA, 200 mM sucrose; pH 8.0). 300 μl lysozyme (˜100,000 U) were added and the mixture was incubated at 37° C. for 30 min, followed by addition of 280 μl of a 10% (w/v) SDS solution and another incubation for 10 min. RNA was digested at room temperature by addition of 240 μl of an EDTA solution (0.5 M, pH 8), 20 μl Tris-HCl (1 M, pH 7.5), and 10 μl RNase A (Fermentas). Then, 100 μl Proteinase K (0.5 U) were added and proteolysis took place for 1-3 h at 37° C. Finally, 600 μl of sodium perchlorate (5 M) were added, followed by a phenol-chloroform extraction and an isopropanol precipitation. DNA quantity and quality was inspected spectrophotometrically. The amplified 573 bp promoter P_{WL} was cloned into the E. coli-Clostridium shuttle vector pMTL 83151 (GenBank accession number FJ797647; Heap et al., 2009) using NotI and NdeI restriction sites and E. coli strain DH5α-T1^{R} (Invitrogen), resulting in plasmid pMTL83157. Since the coding sequence of AOR1 contains two internal NdeI sites, splice overlapping (SOE) PCR (Warrens et al., 1997) was used to remove these NdeI sites without alteration of the codons. Both the 1849 bp PCR product of AOR1 and plasmid pMTL83157 were digested with NdeI and EcoR1, and ligated to produce plasmid pMTL83157-AOR1 (Seq. ID. 17). The insert and promoter of the expression plasmid pMTL83157-AOR1 were completely sequenced using oligonucleotides given in Table 4 and results confirmed that the two internal NdeI sites of AOR1 were successfully altered and they were free of mutations (FIG. 3).

TABLE 4
Oligonucleotides for cloning

| | Oligo- | | SEQ |
|---|---|---|---|
| | nucleotide | | ID |
| Target | Name | DNA Sequence (5′ to 3′) | NO. |
| P_{WL} | P_{WL}-NotI-F | AAGCGGCCGCAGATAGTCATAA | 18 |
| | | TAGTTCC | |
| P_{WL} | P_{WL}-NdeI-R | TTCCATATGAATAATTCCCTCCT | 19 |
| | | TAAAGC | |
| AOR1 | AOR1-NdeI-F | AATTCATATGTATGGTTATGATG | 20 |
| | | GTAAAGTATTAAG | |
| AOR1 | AOR1-SOE-B1 | CTAAATCATAAGAACCACAGTCA | 21 |
| | | GATCC | |
| AOR1 | AOR1-SOE-C1 | CTGACTGTGGTTCTTATGATTTAG | 22 |
| | | ATGC | |
| AOR1 | AOR1-SOE-B2 | CCTGTATTCCCCTTGGATCATAA | 23 |
| | | GC | |
| AOR1 | AOR1-SOE-C2 | GCTTATGATCCAAGGGGAATACA | 24 |
| | | GG | |
| AOR1 | AOR1-EcoR1-R | CTAGAATTCCGAATCAAACTAG | 25 |
| | | AACTTACC | |

TABLE 5
Oligonucleotides for sequencing

| Oligonucleotide | | SEQ ID |
|---|---|---|
| Name | DNA Sequence (5′ to 3′) | NO. |
| M13F | TGTAAAACGACGGCCAGT | 26 |
| M13R | CAGGAAACAGCTATGACC | 27 |
| AOR1-NdeI-F | AATTCATATGTATGGTTATGATG | 20 |
| | GTAAAGTATTAAG | |
| adhE1-F | ATGTGGACAAAGTTACAAAAGTT | 28 |
| | CTTGAGGAAC | |
| adhE1-R | GTAAATATTCAAATATCAACTTT | 29 |
| | ACTGCTTCAAGGGC | |

### Overexpression of AOR1 in Clostridium Autoethanogenum DSM10061:

Plasmids pMTL83157 and pMTL83157-AOR1 were introduced into C. autoethanogenum DSM10061 as described above. C. autoethanogenum transformants were selected using 7.5 μg/mL thiamphenicol. Colonies were observed after 3 days of incubation and they were restreaked onto the same selective agar media for purification. To check the identity of the transconjugants, PCR was carried to detect adhE1 (CAETHG_{—}3747) of C. autoethanogenum DSM10061 using primers adhE1-F (Seq. ID. No. 28; ATGTGGACAAAGTTACAAAAGTTCTTGAGGAAC) and adhE1-R (Seq. ID. No. 29; GTAAATATTCAAATATCAACTTTACTGCTTCAAGGGC). FIG. 4 shows the presence of the expected 576 bp product in both plasmid control and AOR1 overexpression strains. Furthermore, plasmid DNA was extracted from C. autoethanogenum transformants and transformed back into E. coli XL1-Blue MRF′ (Stratagene) before plasmid restriction digest analysis was carried out. This is commonly referred to as ‘plasmid rescue’ because plasmids isolated from Clostridia are not of sufficient quality for restriction digest analysis. FIG. 5 shows the presence of the expected fragments following NdeI and KpnI digestions of rescued plasmids from pMTL83157-AOR1 transformants.

### Overexpression of AOR1 Improves Growth Rate:

The ability of C. autoethanogenum plasmid control (pMTL83157) and AOR1 overexpression strains (pMTL83157-AOR1) to grow autotrophically in 100% CO was tested in triplicates of 250 mL serum bottles containing 50 mL PETC media (Table 3) and pressurized with 30 psi CO. Thiamphenicol was supplemented to a final concentration 7.5 μg/mL for the two plasmid harbouring strains. 400 μL of active culture was inoculated into each serum bottle and liquid phase samples were harvested for OD measurements at a wavelength of 600 nm and metabolite analysis by HPLC.

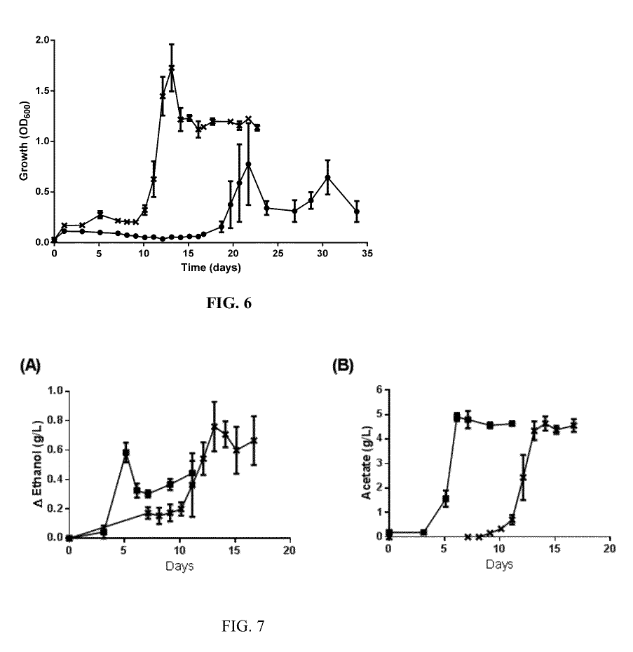

FIG. 6 shows that the overexpression of the enzyme AOR1 catalysing a rate limiting pathway reaction improves autotrophic growth of C. autoethanogenum DSM10061 relative to a plasmid control under 100% CO conditions. For instance, the AOR1 overexpression strain reached a peak OD₆₀₀ of 1.73 on day 13 whereas plasmid control only achieved a peak OD₆₀₀ of 0.78 on day 22.

### Overexpression of AOR1 Increases Ethanol Production of Clostridium Autoethanogenum

In addition, in 100% CO, AOR1 overexpression strain of C. autoethanogenum reached very similar OD₆₀₀ of 1.7-1.8 as the C. autoethanogenum wild-type strain, but the AOR1 overexpression strain of C. autoethanogenum generated 31% more ethanol (FIG. 7A, squares, upper line at day 10) than the wild-type (crosses, lower line at day 10). Acetate production was similar between the recombinant microorganism (FIG. 7B, squares, lower line at day 10) and the wild-type microorganism (FIG. 7B, crosses, upper line at day 10), therefore the AOR overexpression strain produced around 30% higher overall product titers.

In summary, the above example shows how the inventors have successfully demonstrated how to firstly identify rate-limiting pathway reactions and the associated enzymes/co-factors involved in that reaction. Secondly the inventors have produced a recombinant microorganism in which the enzyme exhibits increased activity thus greatly increasing the rate of flux (and hence overall efficiency) through the fermentation pathway.

The invention has been described herein, with reference to certain preferred embodiments, in order to enable the reader to practice the invention without undue experimentation. However, a person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. Titles, headings, or the like are provided to enhance the reader's comprehension of this document, and should not be read as limiting the scope of the present invention.

The entire disclosures of all applications, patents and publications, cited above and below, if any, are hereby incorporated by reference. However, the reference to any applications, patents and publications in this specification is not, and should not be taken as, an acknowledgment or any form of suggestion that they constitute valid prior art or form part of the common general knowledge in any country of the world.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

We claim as our invention:

1. A method of producing a fermentation product, the method comprising:
a) determining a rate-limiting pathway reaction in a fermentation pathway;
b) identifying at least one enzyme, co-factor or both, which are involved in catalysing the rate-limiting pathway reaction;
c) fermenting a CO-comprising substrate with a recombinant carboxydotrophic Clostridia microorganism adapted to exhibit at least one of: i) increased activity of the at least one enzyme of (b) or a functionally equivalent variant thereof, or ii) increased availability of the at least one co-factor of (b), when compared to a parental microorganism, to produce a fermentation product.

2. A method of producing a fermentation product, the method comprising fermenting a CO-comprising substrate with a recombinant carboxydotrophic Clostridia microorganism to produce a fermentation product, wherein the recombinant microorganism is adapted to exhibit at least one of:
a) increased activity of at least one enzyme identified as being involved in catalysing a rate-limiting pathway reaction of a fermentation pathway, or a functionally equivalent variant thereof, when compared to a parental microorganism; or
b) increased availability of at least one co-factor identified as being involved in catalysing a rate-limiting pathway reaction of a fermentation pathway, when compared to a parental microorganism.

3. A method of producing a recombinant carboxydotrophic Clostridia microorganism adapted to exhibit increased flux through a fermentation pathway relative to a parental microorganism, the method comprising:
a) determining a rate limiting pathway reaction in the fermentation pathway;
b) identifying at least one enzyme, co-factor or both, which are involved in catalysing the rate-limiting pathway reaction;
c) transforming a parental microorganism to yield a recombinant microorganism adapted to exhibit at least one of i) increased activity of the at least one enzyme of (b) or a functionally equivalent variant thereof, or ii) increased availability of the at least one co-factor of (b), when compared to a parental microorganism;
wherein the fermentation pathway is capable of producing at least one fermentation product from a substrate comprising CO.

4. A recombinant carboxydotrophic Clostridia microorganism produced by the method of claim 3.

5. A recombinant carboxydotrophic Clostridia microorganism adapted to exhibit at least one of
a) increased activity of at least one enzyme or a functionally equivalent variant thereof when compared to a parental microorganism;
b) increased availability of at least one co-factor when compared to a parental microorganism; or
c) both a) and b);
wherein the at least one enzyme or co-factor have been identified as being involved in catalysing a rate-limiting pathway reaction.

6. The recombinant microorganism of claim 5 wherein the rate-limiting pathway reaction is identified by comparing the enzymatic activity of two or more pathway reactions in the fermentation pathway and selecting the one with the lowest enzymatic activity.

7. The recombinant microorganism of claim 5 wherein the microorganism is adapted to:
i) over-express the at least one enzyme identified as being involved in catalysing a rate-limiting pathway reaction or a functionally equivalent variant thereof;
ii) express at least one exogenous enzyme identified as being involved in catalysing a rate-limiting pathway reaction; or
iii) both i) and ii).

8. The recombinant microorganism of claim 5 wherein the microorganism has undergone enzyme engineering to increase the activity of the at least one enzyme or increase the availability of the at least one co-factor.

9. The recombinant microorganism of claim 5 wherein the microorganism is adapted to exhibit an increase in efficiency of the fermentation pathway relative to the parental microorganism.

10. The recombinant microorganism of claim 9 wherein the increase in efficiency comprises an increase in the rate of production of a fermentation product.

11. The recombinant microorganism of claim 10 wherein the fermentation product is selected from the group consisting of ethanol, butanol, isopropanol, isobutanol, higher alcohols, butanediol, 2,3-butanediol, succinate, isoprenoids, fatty acids, biopolymers, and mixtures thereof.

12. The recombinant microorganism of claim 5 wherein the rate-limiting pathway reaction is present in the Wood-Ljungdahl, ethanol or 2,3-butanediol fermentation pathway.

13. The recombinant microorganism of claim 5 wherein the at least one enzyme is selected from the group consisting of alcohol dehydrogenase (EC 1.1.1.1), aldehyde dehydrogenase (acylating) (EC 1.2.1.10), formate dehydrogenase (EC 1.2.1.2), formyl-THF synthetase (EC 6.3.2.17), methylene-THF dehydrogenase/formyl-THF cyclohydrolase (EC:6.3.4.3), methylene-THF reductase (EC 1.1, 1.58), CO dehydrogenase/acetyl-CoA synthase (EC 2.3.1.169), aldehyde ferredoxin oxidoreductase (EC 1.2.7.5), phosphotransacetylase (EC 2.3.1.8), acetate kinase (EC 2.7.2.1), CO dehydrogenase (EC 1.2.99.2), and hydrogenase (EC 1.12.7.2).

14. The recombinant microorganism of claim 5 wherein the at least one enzyme is selected from the group consisting of pyruvate:ferredoxin oxidoreductase (Pyruvate synthase) (EC 1.2.7.1), pyruvate:formate lyase (EC 2.3.1.54), acetolactate synthase (EC 2.2.1.6), acetolactate decarboxylase (EC 4.1.1.5), 2,3-butanediol dehydrogenase (EC 1.1.1.4), primary:seconday alcohol dehydrogenase (EC 1.1.1.1), formate dehydrogenase (EC 1.2.1.2), formyl-THF synthetase (EC 6.3.2.17), methylene-THF dehydrogenase/formyl-THF cyclohydrolase (EC:6.3.4.3), methylene-THF reductase (EC 1.1, 1.58), CO dehydrogenase/acetyl-CoA synthase (EC 2.3.1.169), CO dehydrogenase (EC 1.2.99.2), and hydrogenase (EC 1.12.7.2).

15. The recombinant microorganism of claim 5 wherein the microorganism is adapted to express an exogenous nucleic acid, or over-express an endogenous nucleic acid involved in the biosynthesis of the at least one enzyme or co-factor involved in catalysing the rate limiting pathway reaction.

16. The recombinant microorganism of claim 5 wherein the recombinant microorganism is adapted to exhibit increased availability of the at least one co-factor.

17. The recombinant microorganism of claim 16 wherein the at least one co-factor is tetrahydrofolate (THF).

18. The recombinant microorganism of claim 17 wherein the microorganism exhibits increased expression of at least one of GTP cyclohydrolase I (EC 3.5.4.16), alkaline phosphatase (EC 3.1.3.1), dihydroneopterin aldolase (EC 4.1.2.25), 2-amino-4-hydroxy-6-hydroxymethyldihydropteridine diphosphokinase (EC 2.7.6.3), dihydropteroate synthase (2.5.1.15), dihydropteroate synthase (EC 2.5.1.15), dihydrofolate synthase (EC 6.3.2.12), folylpolyglutamate synthase (6.3.2.17), dihydrofolate reductase (EC 1.5.1.3), thymidylate synthase (EC 2.1.1.45), or dihydromonapterin reductase (EC 1.5.1.-).

19. The recombinant microorganism of claim 16 wherein the at least one co-factor is cobalamine (B₁₂).

20. The recombinant microorganism of claim 19 wherein the recombinant microorganism exhibits increased expression of at least one of 5-aminolevulinate synthase (EC 2.3.1.37), 5-aminolevulinate:pyruvate aminotransferase (EC 2.6.1.43), adenosylcobinamide kinase/adenosylcobinamide-phosphate guanylyltransferase (EC 2.7.1.156/2.7.7.62), adenosylcobinamide-GDP ribazoletransferase (EC 2.7.8.26), adenosylcobinamide-phosphate synthase (EC 6.3.1.10), adenosylcobyric acid synthase (EC 6.3.5.10), alpha-ribazole phosphatase (EC 3.1.3.73), cob(I)alamin adenosyltransferase (EC 2.5.1.17), cob(II)yrinic acid a,c-diamide reductase (EC 1.16.8.1), cobalt-precorrin 5A hydrolase (EC 3.7.1.12), cobalt-precorrin-5B (C1)-methyltransferase (EC 2.1.1.195), cobalt-precorrin-7 (C15)-methyltransferase (EC 2.1.1.196), cobaltochelatase CobN (EC 6.6.1.2), cobyrinic acid a,c-diamide synthase (EC 6.3.5.9/6.3.5.11), ferritin (EC 1.16.3.1), glutamate-1-semialdehyde 2,1-aminomutase (EC 5.4.3.8), glutamyl-tRNA reductase (EC 1.2.1.70), glutamyl-tRNA synthetase (EC 6.1.1.17), hydroxymethylbilane synthase (EC 2.5.1.61), nicotinate-nucleotide-dimethylbenzimidazole phosphoribosyltransferase (EC 2.4.2.21), oxygen-independent coproporphyrinogen III oxidase (EC 1.3.99.22), porphobilinogen synthase (EC 4.2.1.24), precorrin-2 dehydrogenase/sirohydrochlorin ferrochelatase (EC 1.3.1.76/4.99.1.4), precorrin-2/cobalt-factor-2 C20-methyltransferase (EC 2.1.1.130/2.1.1.151), precorrin-3B synthase (EC 1.14.13.83), precorrin-3B C17-methyltransferase (EC 2.1.1.131), precorrin-4 C11-methyltransferase (EC 2.1.1.133), precorrin-6X reductase (EC 1.3.1.54), precorrin-6Y C5,15-methyltransferase (EC 2.1.1.132), precorrin-8W decarboxylase (EC 1.-.-.-), precorrin-8X methylmutase (EC 5.4.1.2), sirohydrochlorin cobaltochelatase (EC 4.99.1.3), threonine-phosphate decarboxylase (EC 4.1.1.81), uroporphyrinogen decarboxylase (EC 4.1.1.37), and uroporphyrinogen III methyltransferase/synthase (EC 2.1.1.107/4.2.1.75).
